(19) United States
(12) Patent Application Publication
(10) Pub. No.: US 2025/0324965 A1
(43) Pub. Date: Oct. 23, 2025

(54) Indicator Clearance Monitoring in Machine Perfusion of an Organ

(71) Applicant: The Medical College of Wisconsin, Inc., Milwaukee, WI (US)

(72) Inventor: Joohyun Kim, Brookfield, WI (US)

(21) Appl. No.: 18/717,658

(22) PCT Filed: Dec. 1, 2022
(86) PCT No.: PCT/US2022/051504

§ 371 Date: Jun. 7, 2024

Related U.S. Application Data
(60) Provisional application No. 63/287,382, filed on Dec. 8, 2021.

(51) Int. Cl. A01N 1/122; A01N 1/143; C12M 1/34

(52) U.S. Cl. CPC A01N 1/122; A01N 1/143; C12M 41/46

(57) ABSTRACT

A system for monitoring an organ in vitro, comprising: a machine perfusion apparatus for perfusing the organ with a perfusate comprising an indicator; a spectrometer coupled to an input flow cell and an output flow cell, the input flow cell fluidically coupled to a perfusate recirculation input to the machine perfusion apparatus, and the output flow cell fluidically coupled to a physiological fluid output from the organ; and a controller comprising a processor coupled to the input flow cell, the output flow cell, and the spectrometer.

### CROSS-REFERENCE TO RELATED APPLICATIONS

The present application is based on and claims priority from U.S. Patent Application Ser. No. 63/287,382, filed on Dec. 8, 2021, the entire disclosure of which is incorporated herein by reference.

### STATEMENT REGARDING FEDERALLY SPONSORED RESEARCH

N/A

### BACKGROUND

Currently, it is impossible to measure the graft function during organ preservation before liver transplantation (LT) or other organ transplantations. Due to the lack of a surrogate marker, organs with clinical risk factors (i.e., old donor age, fatty change, or increased ischemia time) are often preemptively declined for preventing graft failure. The high organ discard rate has aggravated the organ shortage and waiting list mortality related to organ transplantation including LT.

### SUMMARY

Accordingly, new systems, methods, apparatus, and computer-readable media for determining viability of an organ prior to transplantation are desirable.

Certain embodiments provide a system for monitoring an organ in vitro. The system includes a machine perfusion apparatus for perfusing the organ with perfusate including an indicator; a spectrometer coupled to an input flow cell and an output flow cell, the input flow cell fluidically coupled to a perfusate recirculation input to the machine perfusion apparatus, and the output flow cell fluidically coupled to a physiological fluid output from the organ; and a controller comprising a processor coupled to the input flow cell, the output flow cell, and the spectrometer, the processor being configured to: obtain a first time course of optical measurements from the input flow cell, obtain a second time course of optical measurements from the output flow cell, analyze the first time course of optical measurements and the second time course of optical measurements to identify levels of the indicator, and determine an integrity of the organ in vitro based on identifying the levels of the indicator in the first time course of optical measurements and the second time course of optical measurements.

Other embodiments provide a method for monitoring an organ in vitro. The method includes perfusing, using a machine perfusion apparatus, the organ with perfusate including an indicator; obtaining, using an input flow cell coupled to a spectrometer and fluidically coupled to a perfusate recirculation input to the machine perfusion apparatus, a first time course of optical measurements; obtaining, using an output flow cell coupled to the spectrometer and fluidically coupled to a physiological fluid output from the organ, a second time course of optical measurements; analyzing, using a processor coupled to the spectrometer, the first time course of optical measurements and the second time course of optical measurements to identify levels of the indicator; and determining, using the processor, an integrity of the organ in vitro based on identifying the levels of the indicator in the first time course of optical measurements and the second time course of optical measurements.

Still other embodiments provide a method for assessing a health of a tissue, including: measuring a first level of a first marker in the tissue whose distribution within the tissue changes based on the tissue being damaged; measuring a second level of a second marker in the tissue whose distribution within the tissue does not change based on the tissue being damaged; generating an index based on the first level and the second level; and determining the health of the tissue based on the index.

### BRIEF DESCRIPTIONS OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows a proportion of proteins on the canalicular membrane measured by subcellular protein fractionation.

FIG. 2 shows digital imaging analysis for immunofluorescence staining in a rat liver (green, CD13, reference protein; red, MRP2, canalicular membrane transporter).

FIG. 3 shows a linear correlation between the degree of the membranous proportion of MRP2 (measured by digital imaging analysis) and the degree of liver injury (represented by warm ischemia time).

FIG. 4 shows a fluorescence change in bile after intravenous infusion of fluorescent marker in an in vivo rat model, where the graph shows a control vs. ischemia-reperfusion injury.

FIG. 5A shows schematic diagrams of the overall process.

FIG. 5B shows a diagram of a perfusion system including

FIG. 5C shows a schematic overview of hepatocyte function for normal (left) and ischemia-reperfusion (right) cells. SAC, subapical compartment; HMT, hepatocyte membrane transporters; SF, sodium fluorescein.

FIG. 5D shows HMTs between three compartments involved in bile formation (Blood, Hepatocyte, and Bile).

FIG. 6 shows proportions of liver transplants for liver cancer patients in Wisconsin.

FIG. 7 shows results of all authorized donors for liver transplant in Wisconsin in the year 2020.

FIG. 8 shows a diagram of the dilemma of organ shortages.

FIG. 9 shows a diagram of two targets of HMT function.

FIG. 10 shows HMT mRNA levels at baseline, 60 minutes, and 90 minutes of ischemia time in rats.

FIG. 11 shows HMT expression in pig livers, particularly the effect of subnormothermic rewarming.

FIG. 12 shows HMT mRNA levels in wild type and Nrf2 knockout rats.

FIG. 13 shows alteration of MRP2 staining pattern and the graft outcome after LT (human).

FIG. 14 shows translocation of MRP2 in severe hepatic IRI in rat.

FIG. 15 shows a mechanism of NRF2 activation.

FIG. 16 shows effect of pre-ischemia BARD administration on post-reperfusion cholestasis.

FIG. 17 shows effects of BARD on immunohistochemistry for HMTs.

FIG. 18 shows a diagram of HMT as a therapeutic target and a surrogate marker in hepatic IRI.

FIG. 19 shows different patterns of immunohistochemistry staining for basolateral and canalicular HMTs.

FIG. 20 shows a correlation between the length of warm ischemia time and serum bile acids (24 h post-reperfusion) in rats (Simple linear regression, R²=0.8959, P<0.0001, n=5 per time point).

FIG. 21 shows HMT mRNA levels at baseline, 60 minutes, and 90 minutes of ischemia time in rats.

FIG. 22 changes of MRP3 mRNA levels in pig livers, particularly the effect of subnormothermic rewarming in machine perfusion.

FIG. 23 shows changes of HMT mRNA levels during hepatic IRI.

FIG. 24 shows effects of BARD administration (a single dose, 5 min before ischemia) on post-reperfusion (24 h) cholestasis.

FIG. 25 shows translation of MRP2 in hepatic IRI (rats).

FIG. 26 shows correlation between alteration of HMT staining patterns and graft outcomes after clinical LT.

FIG. 27 shows immunofluorescence colocalization of an HMT (MRP2, red) and a canalicular membrane marker (CD10, green) in a normal rat liver.

FIG. 28 shows three compartments in the bile formation process: A (blood in sinusoid), B (cytosol), and C (bile in the biliary canaliculus); T1: basolateral HMT, T2: canalicular HMT.

FIG. 29 shows comparative analyses of HMT translocation and substrate kinetics.

FIG. 30 shows the study design.

FIG. 31 shows expansion of the study model to a clinical monitoring system in machine perfusion.

FIG. 32 shows a study design. Panel A shows a scheme of five experimental groups. All rats underwent intravenous injection of sodium fluorescein followed by a series of blood and bile samplings over 60 min to measure fluorescence. The rats in all groups, except the controls, underwent partial (70%) ischemia-reperfusion injury (IRI), starting with brief (30 min) or prolonged (60 min) ischemia, followed by 1-h or 4-h reperfusion, before sodium fluorescein injection. Panel B shows photos demonstrating vascular and biliary cannulation. Sodium fluorescein was administered through the right jugular vein. At each designated time point after the administration (0, 2, 5, 10, 20, 30, 45, and 60 min), blood and bile samples were collected from the left carotid artery and the bile duct, respectively. WIT, warm ischemia time. Panel C shows a diagram of the surgical setup.

FIG. 33 shows data from whole blood, plasma, and bile in the control group. Panel A shows a time course of fluorescence intensity changes, demonstrating overall differences between bile (green triangle), plasma (blue squares), and whole blood (red circles) from samples in the control group (n=5). Values are presented as medians with interquartile ranges. Panel B shows a scatter plot demonstrating correlation between fluorescence intensity values from whole blood and plasma samples from 5 rats in the control group (n=40; samples from 8 time points per animal). The correlation was determined by the Spearman's rank correlation coefficient (R).

FIG. 34 shows fluorescence time courses determined from whole blood, plasma, and bile samples in three warm ischemia time (WIT) groups: 0 min (control, green triangles), 30 min (blue squares), and 60 min (red circles). Values are presented as medians with interquartile ranges. Effects of WIT were determined by repeated measures ANOVA with the Geisser-Greenhouse correction. Panel A shows data measured after 1 h of reperfusion. Panel B shows data measured after 4 h of reperfusion. n=5 each.

FIG. 35 shows relative fluorescence intensity values of bile to whole blood or plasma values in three warm ischemia time (WIT) groups; 0 min (control, green triangles), 30 min (blue squares), and 60 min (red circles). Values are presented as medians with interquartile ranges. The effects of WIT were determined by repeated measures ANOVA with the Geisser-Greenhouse correction. Panel A shows the bile-to-whole blood (top) and the bile-to-plasma (bottom) fluorescence ratios measured 1 h after reperfusion. Panel B shows bile-to-whole blood (top) and bile-to-plasma (bottom) fluorescence ratios measured 4 h after reperfusion. n=5 each.

FIG. 36 shows bile-to-plasma fluorescence ratios in three warm ischemia time groups (green triangles, 0 min; blue squares, 30 min; red circles, 60 min). Data collected at selected time points (30, 45, and 60 min after sodium fluorescein injection) are presented. Panel A shows data in the control and 1 h postreperfusion groups. Panel B shows data in the control and 4 h postreperfusion groups. n=5 each. Black bars represent median values. The differences among the groups were evaluated by the Kruskal-Wallis test (P values in each panel) and the post hoc Mann-Whitney test with Bonferroni's correction (* between the groups). n=5 each, WIT, warm ischemia time.

FIG. 37A shows the proportion of canalicular membrane CD13 relative to total CD13, determined using immunoblotting. FIG. 38B shows a schematic demonstration of TTI measurements using immunofluorescence images. FIG. 38C shows a time-translocation plot between MRP2 intracytosolic translocation and warm ischemia time (WIT), where n=5 per time point. A simple linear regression was used to fit the line.

FIG. 38 shows human liver transplantation data.

FIG. 39 shows ex vivo rat liver machine perfusion data.

### DETAILED DESCRIPTION

In accordance with some embodiments of the disclosed subject matter, mechanisms (which can include systems, methods, apparatus, and computer-readable media) for determining viability of an organ prior to transplantation are provided.

To develop an effective clinical strategy in addressing the organ shortage, e.g. in liver transplantation (LT), it would be helpful to develop an objective method to assess the viability of livers or other organs during organ preservation and prior to implantation into an organ recipient subject. An opportunity to develop such methods arose when normothermic machine perfusion systems became available in the clinical arena. Current viability criteria during liver machine perfusion rely on biochemical assays of perfusate or bile. These approaches are not only impractical, but also have shown disappointing discriminatory power to predict viable organs for LT. As such, there is no useful method to determine the viability of liver organs for transplantation.

Accordingly, the present disclosure provides embodiments of a real-time portable monitor that can measure the viability of a liver based on fluorescent marker clearance during machine perfusion. The disclosure is based on a hypothesis that the clearance of fluorescent markers mediated by specific membrane transporters of liver cells represents the degree of liver injury during organ preservation. A rationale is that although severe hepatic ischemia-reperfusion injury (IRI) during organ preservation is associated with cholestasis as a result of membrane transporter dysfunction, a rapid and precise assessment that quantifies the functional capacity of membrane transporters is unavailable. In various embodiments the disclosed procedures will also enhance a widely applicable platform for studying cholestatic liver disease, and in further embodiments may be used for assessing drug metabolism in pharmaceutical industries.

The disclosure provides various embodiments of novel diagnostic devices, methods, systems, and computer-readable media that can measure organ functionality (e.g. the integrity of bile metabolism) in real time and that can be attached or adapted to a portable machine perfusion system for organs (e.g. liver organs). The mechanistic background is as follows, which is described in terms of assessing liver function through monitoring of bile metabolism; nevertheless, in various embodiments the same underlying principles may be applied to other tissues and organs besides the liver.

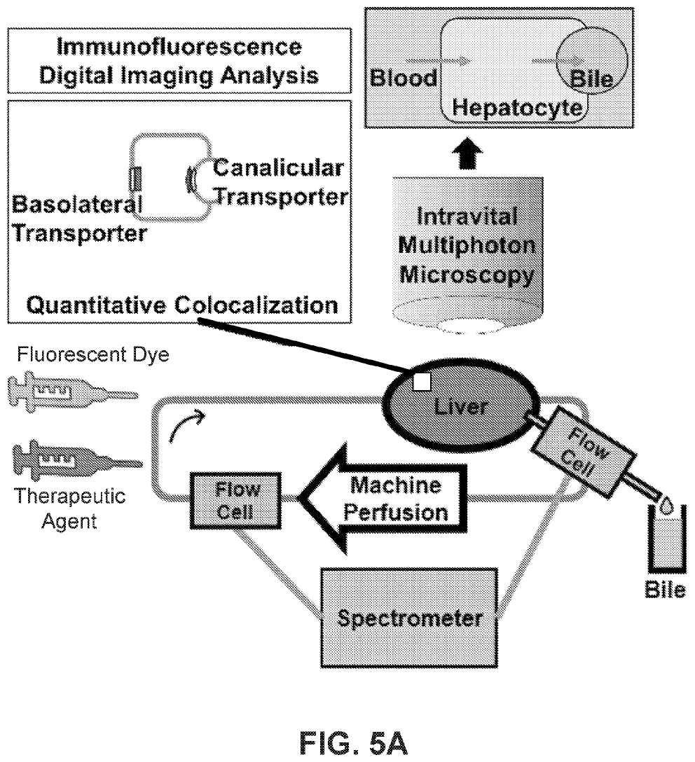

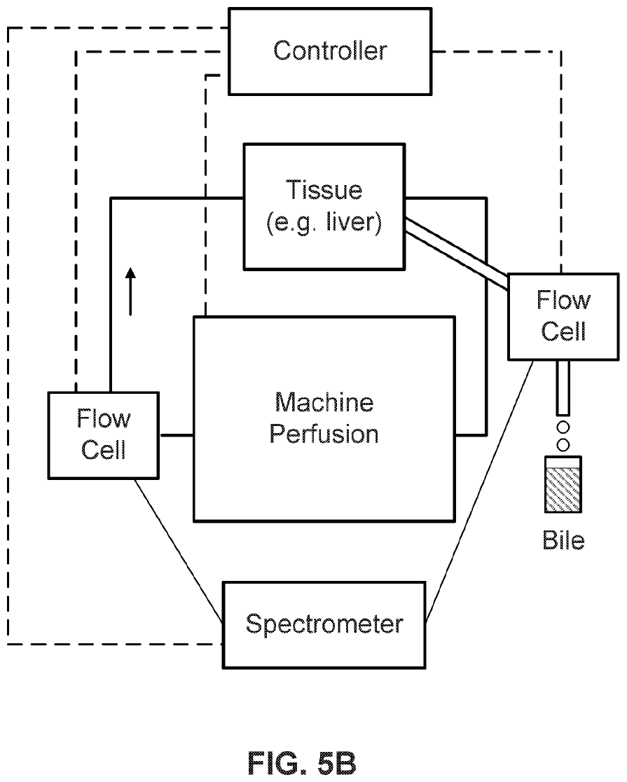

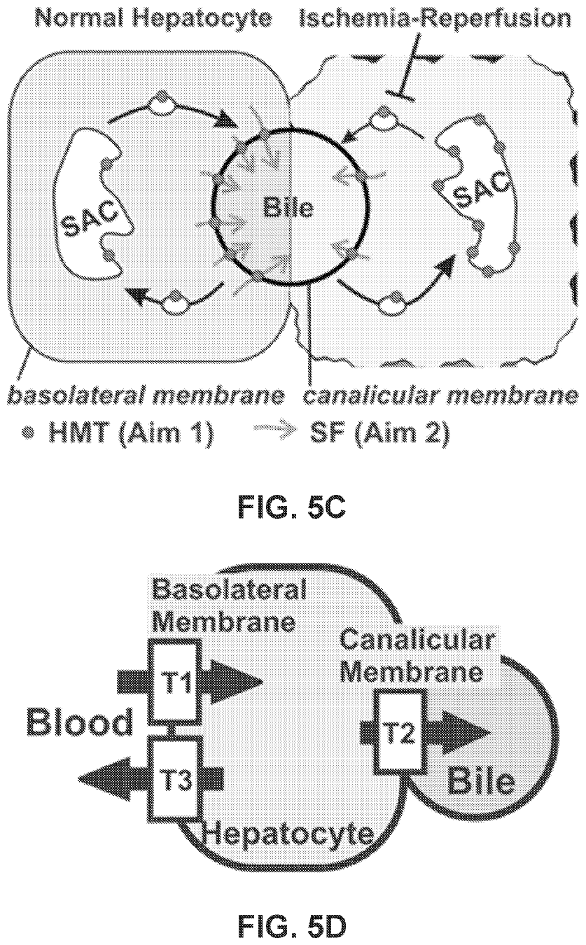

Bile metabolism, as the core biological function of the liver, reflects liver viability in LT. Bile formation is a function of basolateral and canalicular membrane transporter proteins in hepatocytes (see FIGS. 5C, 5D). FIG. 5C shows a schematic overview of hepatocyte function for normal cells (left) and ischemia-reperfusion cells (right). As shown in FIG. 5C, in a normal hepatocyte the hepatocyte membrane transporters (HMTs) are present in the membrane leading to the bile ducts where they help transport substances (e.g. sodium fluorescein, SF, under experimental conditions) to be excreted into the bile, whereas relatively fewer of the HMTs are present in the subapical compartment (SAC). In injured hepatocytes, HMT transporter proteins are located primarily at cell membranes and thus measuring locations of HMTs provides an indicator of the stress level of the liver tissue. While the HMTs are shuttled between the bile duct and the SAC, in the ischemia-reperfusion cells the transfer of HMTs from the SAC to the bile duct is inhibited and as a result HMTs are present in lower numbers in the bile duct and higher numbers in the SAC. FIG. 5D shows HMTs between three compartments involved in bile formation, namely a blood vessel, hepatocytes, and the bile duct. The HMTs, which are labeled T1, T2, and T3, can transport material to and from the blood vessel (T1 and T3) as well as into the bile duct (T2).

Intriguingly, certain fluorescent dyes are metabolized by specific membrane transporters and excreted into bile. For example, sodium fluorescein is metabolized by the liver through OATP1B2 and MRP2, and indocyanin green (ICG) through OATP1B2, NTCP, and MDR2, a process which occurs within 15 minutes in a healthy liver. As such, the distribution and clearance kinetics of fluorescent dyes between the blood, hepatocytes, and bile represent the function of basolateral and canalicular transporters on the hepatocyte cell membrane. Various embodiments involve monitoring the liver in machine perfusion after the injection of fluorescent markers, individually or in combination (e.g., a mixture of sodium fluorescein and ICG), through simultaneous real-time monitoring using fluorescence spectroscopy attached to the circuit and the biliary drain through flow cells (FIG. 5A). As such, the clearance of fluorescent markers for basolateral and canalicular transporters of liver cells can be measured by measuring specific fluorescence emission in perfusate and in bile, and the clearance is taken as an indicator of the viability of the liver tissue. In some embodiments, a fluorescent dye and/or a therapeutic agent can be added to the perfusion system, where the dye can be used to track the viability of the tissue and the therapeutic agent can be used to stabilize or improve the condition of the tissue or to help assess the condition of the tissue (e.g. in conjunction with the fluorescent dye). Therapeutic agents may include one or more of de-fatting agents, agents for gene silencing with RNA interference, immunomodulation agents, or anti-inflammatory agents, or other agents such as those described in Dengu et al. (“Normothermic Machine Perfusion (NMP) of the Liver as a Platform for Therapeutic Interventions during Ex-Vivo Liver Preservation: A Review,” J. Clin. Med., 9:1046 (2020)), incorporated herein by reference in its entirety.

In certain embodiments, an existing perfusion device may be adapted to introduce one or both of a fluorescent dye and/or a therapeutic agent to the perfusate and to monitor fluorescence levels in the perfusate and/or bile, e.g. by adapting the device to include a spectrometer and one or more flow cells as diagrammed in FIG. 5A. In addition, an imaging device (e.g. an intravital multiphoton microscopy system as in FIG. 5A or other suitable imaging system) may be adapted to the perfusion system to monitor the tissue, for example to directly visualize fluorescent material in the hepatocytes, blood vessels, and/or bile ducts. In healthy liver tissue, direct measurements of the fluorescence levels in the tissue should be seen decrease over the time course of the study, however, at present it is simpler to monitor fluorescence levels in the bile and perfusate as shown in the diagram in the lower portion of FIG. 5A to obtain an indirect indication of the fluorescence levels in the liver tissue.

In various embodiments, experimental data obtained during perfusion and/or from subsequent transplant may be incorporated into a computational model of liver transplant. The computational model may include steps of data interpretation and refinement as well as assessment of hepatic ischemia-reperfusion injury (IRI) based on the data.

Thus, in various embodiments an apparatus, method, or system for monitoring an organ in vitro may include a machine perfusion apparatus, a spectrometer, and a controller (FIG. 5B). The machine perfusion apparatus may perfuse the organ with a perfusate and the perfusate may include an indicator substance. The spectrometer may be coupled to an input flow cell and an output flow cell. The input flow cell of the spectrometer may be fluidically coupled to a perfusate recirculation input to the machine perfusion apparatus. The output flow cell of the spectrometer may be coupled to a physiological fluid output from the organ (e.g. a bile duct of the liver).

In certain embodiments, the controller may include a processor and the controller and/or processor may be connected (e.g. in a wired or wireless manner) to the input flow cell, the output flow cell, and the spectrometer in a manner that permits bidirectional communication (e.g. to send and receive control signals and/or data between the devices). In various embodiments, the processor may be configured to carry out a procedure for monitoring an organ in vitro. This procedure may include obtaining a first time course of optical measurements from the input flow cell and a second time course of optical measurements from the output flow cell, where a time course may include multiple (e.g. two or more) optical measurements that are obtained at at least two different points in time. The input flow cell may obtain data at the same time as the output flow cell such that the first time course and the second time course overlap in time and may include groups of data values (i.e. one or more value from the input flow cell and one or more value from the output flow cell) at each time point. The procedure may also include analyzing the first and second time courses of optical measurements to identify levels of the indicator substance during each of the respective time courses. The procedure may include determining the integrity of the organ based on identifying levels of the indicator substance in each of the first and second time courses.

The organ may be determined to be healthy if there is evidence that the indicator substance is being metabolized and/or transported by the organ. For example, if the levels of the indicator substance in the perfusate decrease during the time course or are at or below an input target level then this may be taken as an indication that the organ is healthy since it indicates that the organ is removing the indicator substance from the perfusate, e.g. by metabolizing or transporting the indicator substance. If levels of the indicator substance in the output of the organ increase during the time course or are detected at or above an output target level then this may be taken as an indication that the organ is healthy. Each of the organ perfusate and organ output levels of indicator substance levels may be assessed independently or in conjunction with the other when determining organ health/viability. For example, the levels of indicator substance from the organ output measurements may be normalized by the levels of indicator substance in the organ perfusate before assessing the trend or absolute level of the measurements.

In certain embodiments the organ that is being evaluated is a liver, however in other embodiments the organ may be another internal organ (generally an organ with a secretory function) such as a kidney. When the organ is a liver, the output flow cell may be coupled to the bile duct of the liver such that the optical measurements obtained from the output flow cell are indicative of substances that are contained in the bile excreted by the liver. The indicator substance may include fluorescein, indocyanin green (ICG), rhodamine-123, DY635, C-Carboxyfluorescein diacetate, cholyl-lysl, fluorescein, or cholyl-glycyl-amido-fluorescein, each of which may be transported by molecules in liver cells. When the indicator substance is a fluorescent molecule such as fluorescein or ICG, the spectrometer may be configured to obtain fluorescence measurements from the material within the first and second flow cells such that the first and/or second time courses of optical measurements include fluorescence measurements.

In various embodiments, the procedures may include coupling an imaging system to the organ to monitor and evaluate the organ structurally. In particular embodiments the imaging system may include a multiphoton microscopy system (e.g. an intravital multiphoton microscopy system), which provides good depth resolution within the organ and which is minimally invasive.

In some embodiments, the procedures may include a method for monitoring an organ in vitro. The method may include perusing the organ with a perfusate that includes an indicator substance, where the organ is perfused using a machine perfusion apparatus. The method may also include a first time course and a second time course of optical measurements. The first time course may be obtained using an input flow cell coupled to a spectrometer and fluidically coupled to a perfusate recirculation input to the machine perfusion apparatus. The second time course may be obtaining using an output flow cell coupled to the spectrometer and fluidically coupled to a physiological fluid output from the organ. The first and second time courses of optical measurements may be analyzed using a processor that is coupled to the spectrometer to identify levels of the indicator. The integrity of the in vitro organ may be determined by the processor based on identifying the levels of the indicator in the first time course and the second time course of optical measurements. Certain embodiments may include a computer-readable medium which includes instructions for carrying out the disclosed methods.

Some embodiments provide procedures (e.g. systems, methods, apparatus, and/or computer-readable media) for assessing a health of a tissue, as shown below in Example 5, using cell markers (which can be used alone or in conjunction with information from a perfusion system) to assess quality of liver tissue prior to transplantation. The procedure may include measuring levels of a first marker in the tissue whose distribution within the tissue changes based on the tissue being damaged and a second marker in the tissue whose distribution within the tissue does not change based on the tissue being damaged. The distribution of the second marker can be used as a reference to normalize the levels of the first marker, particularly in the case of a marker such as CD13 which remains distributed within the canalicular membrane of the liver.

The procedure may include obtaining samples of the tissue such as liver tissue and staining the samples (e.g. using immunofluorescence) for the first and second markers and imaging the stained samples (e.g. using fluorescence microscopy). The relative levels and distributions of the first and second markers are then used to generate an index which can be used to determine the health of the tissue.

In the case where the tissue is liver tissue, the first marker can be a transporter molecule such as MRP2 and the second marker can be a marker that is distributed in the canalicular membrane such as CD13. The procedure can include determining a first area in the sample covered by MRP2 and CD13 and determining an area covered by only MRP2 by subtracting regions of overlap between MRP2 and CD13 and dividing this area by the total area covered by all MRP2 to determine the index. As the CD13 marker is primarily distributed in the canalicular membrane, the area covered only by MRP2 represents cytosolic MRP2. Therefore, the index (which may be referred to as the Transporter Translocation Index, TTI) represents the cytosolic distribution of MRP2 within the tissue. Given that MRP2 redistributes from the canalicular membrane to the cytosol in response to liver tissue damage, the index of MRP2 distribution can be taken as a measure of the health of the tissue. The index value may be compared to a reference index value or to a cutoff value to assess the health of the liver tissue. The reference or cutoff values may in turn be determined by comparison with known markers such as alanine aminotransferase (ALT) levels in patients who have received liver transplants. A body of data may be developed to relate index values to transplant success (e.g. using various indicators including ALT levels) to develop the reference or cutoff values. The index value may be determined based on a single tissue sample or a series of index values may be determined over time, for example based on biopsies obtained at various stages including: prior to removal of the tissue from a donor, during transport of the donated tissue, and/or after implantation of the donated tissue into a recipient.

The following are a series of non-limiting Examples which describe experiments designed to evaluate the use of the procedures to monitor organs in vitro, focusing on the liver.

### EXAMPLES

### Example 1

This Example's objective is to determine the feasibility of using real-time fluorescence monitoring to evaluate the viability of the liver in ex vivo machine perfusion using a rat hepatic ischemia-reperfusion injury (IRI) model. A goal is to develop a novel diagnostic device that can measure the integrity of the bile metabolism in real time that can be attached to a portable machine perfusion system for organs for liver transplantation (LT). Traditionally, static cold storage has been the standard organ preservation technique for LT over the past five decades because of its simplicity and cost effectiveness. However, the static method does not allow the monitoring of graft viability. An opportunity arose when normothermic machine perfusion systems became clinically available. The novel method entails placing a liver graft in a machine perfusion circuit with oxygenated blood. The system simulates a physiologic condition, which allows the liver to produce bile. It was expected that biochemical assays of perfusate or bile during machine perfusion might predict outcomes after LT. However, these approaches are not only impractical but also have shown disappointing discriminatory power to predict viable organs for LT.

Notably, our previous clinical and animal studies have shown the association between hepatic IRI and dysfunctional bile formation (i.e., cholestasis). As such, we hypothesized that the mechanistic function underlying bile formation, as the core biological activity of the liver, reflects its viability. Bile formation occurs across two distinct areas of the hepatocyte cell membrane. One is the basolateral membrane between blood and the hepatocyte, and the other is the canalicular membrane that lies between the hepatocyte and bile. The basolateral and canalicular cell membrane encompass a repertoire of membrane transporter proteins to mediate substrate transportation from blood into bile through the hepatocyte. Therefore, bile formation is a function of basolateral (e.g., OATP1B2) and canalicular membrane transporter proteins (e.g., MRP2). Among the two steps, transport across the canalicular membrane is rate-limiting in bile formation, as it requires energy-dependent transit against a 1,000-fold concentration difference. Importantly, transporter proteins exist in a recycling pool for rapid mobilization and insertion between the submembrane vesicle and the cell membrane. Among the various mechanism that can regulate the function of bile transporters, the endocytosis-mediated translocation has been suggested as the main mechanism of cholestasis in acute stress, such as IRI.

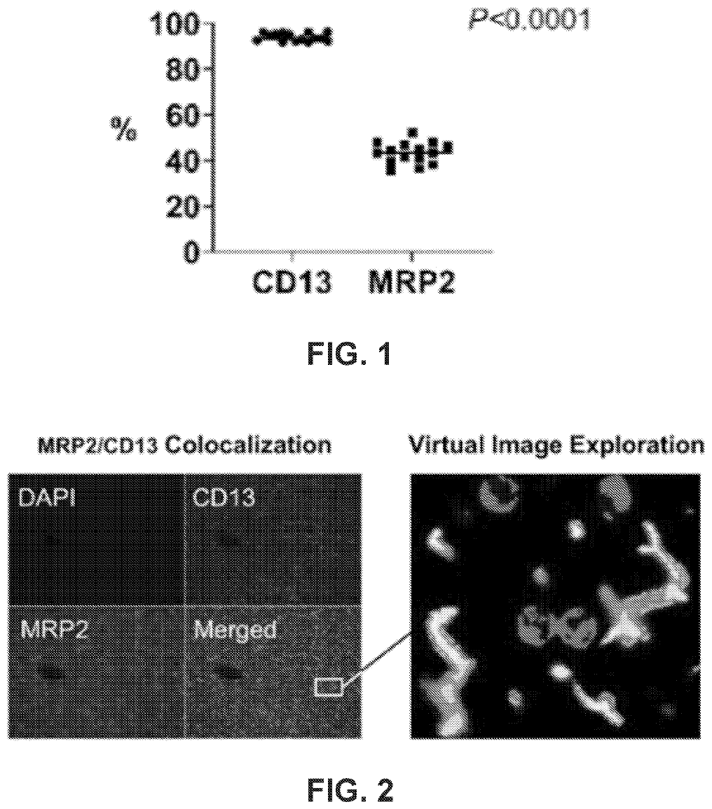

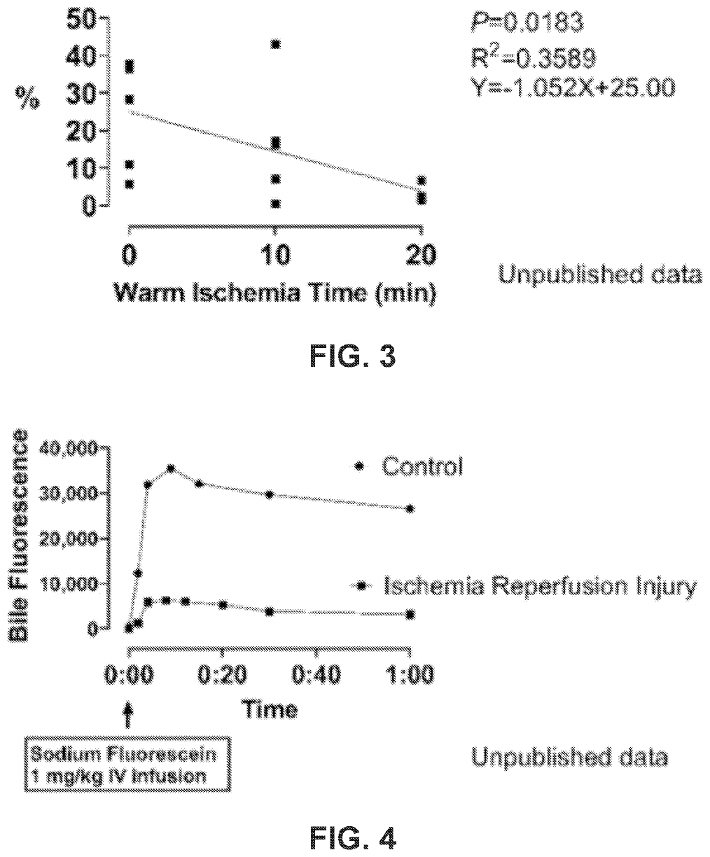

In this regard, if a reference protein can be stained to demarcate the area of basolateral or canalicular membrane, the degree of translocation of a specific transporter protein can be quantified using immunofluorescence colocalization. Recently, we identified CD13 as a marker that demarcates the canalicular membrane area (FIG. 1). Therefore, immunofluorescent colocalization staining can distinguish the membranous portion of a transporter protein from the cytosolic portion of the protein (FIG. 2). Our preliminary experiment to determine the area of translocation showed the size of the MRP2 area relative to CD13 estimated as colocalized was predictive of liver viability, as the degree of translocation was associated with ischemia time (FIG. 3). As the ischemia time is an objective and adjustable indicator of IRI, this model provides an opportunity to develop a tool for monitoring the degree of IRI. By extension, the clearance kinetics of substrates among blood, hepatocytes, and bile may represent the function of basolateral and canalicular transporters. Importantly, certain fluorescent dyes are metabolized by specific membrane transporters and excreted into the bile. For example, fluorescein is transported by the liver through OATP1B2 and MRP2, and the process occurs within 15 minutes in a healthy liver. We tested the concept in our in vivo IRI model using laboratory rats. Excretion of fluorescein measured by fluorescence in bile samples was noticeably compromised by IRI (FIG. 4). As such, the decrease in the clearance of fluorescent markers mediated by specific membrane transporters may represent the degree of hepatic IRI. Based on our preliminary data, we developed a research plan as follows.

### Methods and Strategies

A goal is to develop a real-time monitoring system of bile metabolism by measuring fluorescent dye clearance from the liver in a machine perfusion system to improve viability assessment of donor livers for LT. Fluorescent dyes that are the substrates of relevant membrane transporters in the hepatocyte will be tested, and the clearance of fluorescent dyes among blood, hepatocytes, and bile will be monitored using a fluorescence spectroscopy and multiphoton microscope in our rat liver machine perfusion model. Our approach will elucidate the mechanistic relationship between the translocation of membrane transporters and cholestatic dysfunction, as well as quantify bile metabolism using the clearance of the fluorescent substrates of the transporters in a machine perfusion system, advantages not provided by conventional approaches. We will test our hypothesis and attain our objective through the following two aims:

Aim 1: Determine the underlying pathophysiology of bile transporter dysfunction in hepatic IRI. We will develop a digital imaging analysis approach for CD13 (aminopeptidase N, a reference protein), and MRP2 (multidrug resistance-associated protein 2, the target transporter protein) and quantify the degree of translocation and colocalization using immunofluorescence staining of histologic liver sections. We will test this in various influencing in vivo and ex vivo conditions using rats with conditions related to IRI, such as ischemia time, sex, and age. Working hypothesis: IRI is the key feature that affects tissue viability and the degree of membrane transporters' intracellular translocation will correlate with the degree of injury and will negatively correlate with the clearance of relevant fluorescent markers.

Aim 2: Determine the clearance of fluorescent markers in IRI-affected livers based on machine perfusion. We will monitor the liver in machine perfusion after the injection of fluorescent markers using fluorescence spectroscopy attached to the circuit and the biliary drain through flow cells, simultaneously with investigation under a multiphoton intravital microscope (FIG. 5A). The fluorescence curve can be affected by a variety of factors such as amount of dye, time since injection, back-diffusion, recirculation, concentration, first-pass extraction fraction, or transit time. In this regard, we will develop, parameterize, and validate mechanistic computational models of the disposition of the fluorescent markers on passage through the liver. Model parameters will be estimated using the measured data from the machine perfusion system hemodynamics, volumes of distribution, tissue sample analysis, and clearance parameters. Working hypothesis: The clearance index of the fluorescent markers for membrane transporters based on mechanistic computational modeling will predict the degree of hepatic IRI.

The proximate expected outcome of this work is an integrated understanding of the functional contribution of various membrane transporters in hepatic IRI-related cholestasis. The research is expected to provide an accurate measure of the degree of correlation between the clearance of specific fluorescent markers and the length of ischemia time, which will result in a comprehensive model of the clearance of fluorescent markers in the machine perfusion of livers. The model will allow for a mechanistic and quantitative interpretation of the fluorescent markers' dynamic data, as well as for the estimation of parameters descriptive of the dominant processes that determine the markers' liver uptake, metabolism, and clearance, incorporating the kinetics of membrane transporters. Notably, we have already established our ex vivo perfusion model, and various biological data can be obtained simultaneously from the platform (FIG. 5A). As such, exploratory data analysis and mechanistic computational modeling will be performed during the first year based on immunofluorescence digital imaging, multiphoton microscopy, and real-time fluorimetry, followed by validation studies advanced modeling during the second year.

Scientific Rigor and Statistical Method: To maintain the highest possible Scientific Rigor, biological markers of liver function will be measured with appropriate controls. The number of animals will be determined based on a power analysis while accounting for previous experience and anticipated losses. Experimental conditions and rats will be randomized, and operators will be blinded of the hypothesis being tested. Data will be analyzed using the Mann-Whitney U test, the Kruskal-Wallis H test, or the repeated measures ANOVA, as appropriate. Both male and female rats of the same age will be studied in each protocol. The computational models will be developed by utilizing independently published datasets and data from the studies. The model parameter identifiability and estimability will be evaluated based on parameter sensitivity analysis and correlation coefficient matrix. The team of investigators will meet regularly to evaluate the data and monitor the progress.

### Impact on Public Health

End-stage liver disease (ESLD) is the eighth-leading cause of death in the United States, and the average life expectancy of patients is only two years. The economic burden is substantial, with annual direct costs exceeding $2 billion and indirect costs exceeding $10 billion. Currently, the only cure for ESLD is liver transplantation (LT). As of today, more than 65,000 people in the United States are living with a transplanted liver. However, approximately 12,000 people are still waiting for LT, and thousands of patients die annually while on the waiting list. Since 1995, the number of LTs increased by 202% in Wisconsin, while wait list mortality increased by 538% during the same period. The disproportionate increase in the need for LT compared with the lack of available organs is a serious issue in Wisconsin.

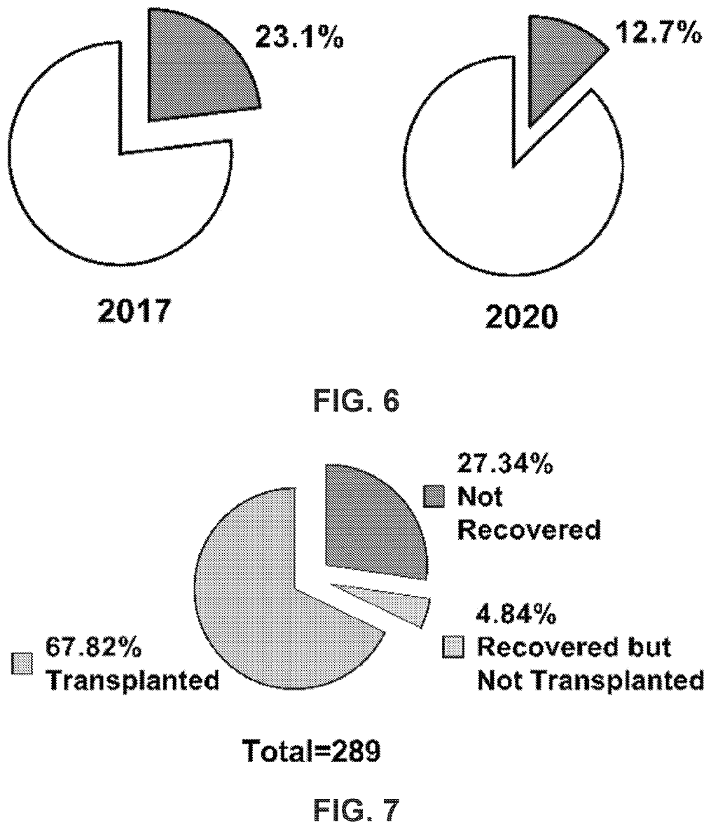

Limited access to LT has also affected liver cancer patients in Wisconsin. Notably, liver cancer is the seventh-leading cause of cancer-related death in Wisconsin. The high rate of morality is associated with liver cirrhosis, which is estimated to occur in up to 90% of liver cancer cases. Concerning cirrhosis with liver cancer, treatment options are significantly limited due to the high incidence of treatment failure and serious complications. LT thus offers the best chance of cancer clearance and restoration of liver function for patients with liver cancer. Liver cancer is therefore an important indication for LT. However, due to the serious shortage of donor organs, access to LT for patients with liver cancer has significantly diminished in the past few years. In 2017, 17.1% of LTs in the United States were provided to liver cancer patients. The organ allocation policy for liver cancer patients has since been restricted, and in 2020, only 11.9% of LTs were provided to liver cancer patients (a 30% decrease). The decrease was even more pronounced in Wisconsin. In 2017, 23.1% of the LTs in Wisconsin were provided to liver cancer patients, falling to 12.7% in 2020 (a 45% decrease, FIG. 6). Importantly, racial disparities with regards to liver cancer mortality in Wisconsin have been noted. A study in 2019 demonstrated that liver cancer mortality was higher among Black residents in a census tract in Wisconsin. Furthermore, it has been reported that patients were less likely to undergo evaluation, waitlisting and liver transplantation if they were women, Black and lacked commercial insurance. As such, providing equal access to health care resources to treat liver cancer should be an important goal, and significant efforts are needed to expand the donor pool to meet the need for LTs in Wisconsin.

To make matters worse, significant numbers of donor organs have been rejected for LT, and organ discard rates remain high despite a growing organ shortage. The liver discard rate in LT in the United States is about 10% annually for all recovered livers for transplantation. The reluctance to use grafts of marginal quality stems from fear of graft failure, which occurs after LT in about 5-8% of cases. The number of unused organs for LT is significant in Wisconsin. In 2020, of the livers from 289 potential Wisconsin donors who authorized for organ donation, 32% could not be used for transplantation (FIG. 7). To develop an effective clinical strategy to avoid both preemptive organ discards and posttransplant liver failure, it is necessary to develop an objective method to assess the viability of livers during preservation.

Using our distinctive combination of scientific and surgical expertise, we will develop new approaches to measuring the bile metabolism activity in a machine perfusion system, including how it associates with liver viability. The results will have an important positive impact because they will foster a better understanding of the role of membrane transporters in acute cholestasis from IRI. In particular, the results will lay the groundwork for developing an innovative method of liver viability monitoring that is real-time, portable, and precise, qualities required in the rapid assessment of graft function for LT. This research is significant because a novel device developed based on this project can be equipped to future clinical trials regarding machine perfusion for LT, which will impact the health of Wisconsin by providing an opportunity to develop a method to measure quantitatively the viability of organs in LT; therefore, the organ discard rate can be reduced, which will allow more LTs to save the lives of ESLD and liver cancer patients in Wisconsin and beyond.

### Example 2

Currently, it is impossible to measure the graft function during organ preservation before liver transplantation (LT). Due to the lack of a surrogate marker, organs with clinical risk factors (i.e., old donor age, fatty change, or increased ischemia time) are often preemptively declined for preventing graft failure. The high organ discard rate has aggravated the organ shortage and waiting list mortality related to LT. We disclose a radically different method of targeting bile metabolism for monitoring and treating hepatic graft function. The function of hepatocyte membrane transporters (HMTs) is fundamental in this approach as it determines overall bile metabolism. The biological response of HMTs to hepatic ischemia/reperfusion injury (IRI) during organ preservation has not yet gained attention because it is difficult to measure its function in the conventional cold static organ preservation method. Recently, normothermic machine perfusion for the liver graft became available in the clinical arena. Machine perfusion can provide a more physiological condition in which the metabolism of HMTs can be potentially monitored and treated. Accordingly, we will measure the biliary clearance of fluorescent probes of HMTs in machine perfusion for monitoring their metabolism in the liver graft. The function of HMTs is known to be determined by their transcriptional activities and the vesicle-based trafficking on the hepatocyte membrane. As such, it could be enhanced by transcription factor activation for the former and endocytosis inhibition for the latter. We will investigate the following: (1) HMT function monitoring by intravital imaging and dynamic clearance in experimental models using rat and pig livers, and (2) the role of targeted therapy, such as transcription factor activators (e.g., bardoxolone methyl) or endocytosis inhibitors (e.g., glucagon), on bile metabolism in machine perfusion. The objective is to provide fundamental data for a future clinical trial of pharmaceutical intervention in the human liver's HMT function in a machine perfusion system for LT. This work could potentially reduce the thousands of preventable deaths every year related to failure of selection of acceptable organs, which is directly relevant to the mission of the National Institutes of Health. In summary, we will innovate the current LT practice by elucidating molecular events of HMT dysfunction in hepatic IRI, allowing the bile metabolism to be monitored and improved during organ preservation.

### Project Narrative

In this era of donor organ shortage, the selection power of acceptable organs determines the waiting list mortality and the success rate in liver transplantation. We suggest that the bile metabolism represents the graft quality, and not only can this be measured, but it can also be enhanced during organ preservation. This project suggests innovative approaches to monitoring and improving bile metabolism by targeting the relevant proteins collectively known as hepatocyte membrane transporters.

### Project Science Areas: 6 MCB; 5 IE

Project Description

### I. Introduction

### The Organ-Shortage Era

End-stage liver disease is a fatal condition: It is the eighth leading cause of death in the United States, and the average life expectancy is as low as 2 years. The economic burden of liver cirrhosis in the United States is substantial, with annual direct costs exceeding $2 billion and indirect costs exceeding $10 billion. A cure for end-stage liver disease was not available until liver transplantation (LT) was first developed in 1963. Currently, more than 65,000 people are living with a transplanted liver in the United States. However, approximately 12,000 people are still waiting for LT, and over 2,000 patients die annually while on the waiting list. As such, the shortage of donor organs is a critical barrier in the management of patients with end-stage liver disease. Paradoxically, about 1,700 livers from deceased organ donors are discarded each year due to concern about organ quality.

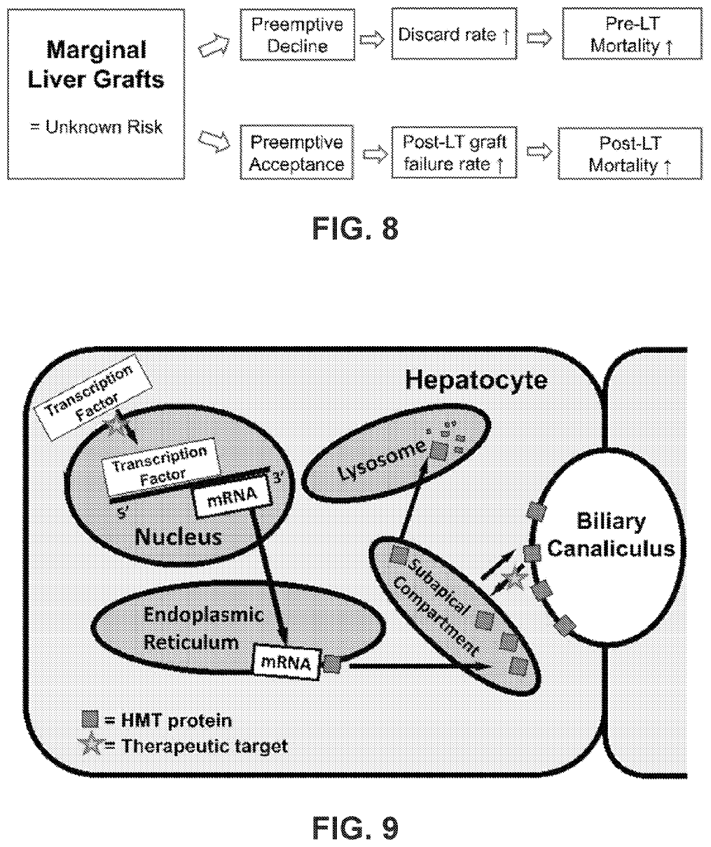

The Irony of Abandoned Livers The numbers of waiting list deaths (2,000/year) and discarded liver organs (1,700/year) are similar. Therefore, unused marginal grafts are a potential resource for addressing organ shortage in LT. The reluctance to use grafts of marginal quality stems from the fear of graft failure, which occurs in approximately 5-8% of cases after LT, even with the high rate of preemptive organ discard. This results in a dilemma in the era of organ shortage: Avoidance of using marginal liver grafts will increase the waiting list mortality, while its encouragement will increase the post-LT mortality from graft failure (FIG. 8). Currently, the decision is based on subjective data, such as the local organ availability and severity of the recipient's condition. For developing a novel clinical strategy, it may be important to understand the two-stage nature of the pathophysiology underlying ischemia/reperfusion injury (IRI).

### IRI: A Two-stage Phenomenon

The LT procedure comprises the donor organ procurement and organ implantation in the recipient. At the time of organ procurement, blood flow in the organ must be interrupted, and tissue ischemia is inevitable. Ongoing ischemic insult to the cells results in the activation of Kupffer cells, the specialized macrophages located in the lining of the liver sinusoids. After reperfusion of the blood flow in the recipient, neutrophils are recruited into the liver parenchyma due to the signal produced by the Kupffer cells. Neutrophils then directly injure hepatocytes via oxidants and enzymes, leading to necrotic cell death. Therefore, the degree of tissue damage is determined by Kupffer cells during ischemia, and the damage is executed by neutrophils during reperfusion.

The final decision on whether to accept a graft for LT should be made during ischemia, at the time of organ preservation. However, a useful test is not available during the organ preservation to determine what choice should be made. As such, the vulnerability of a liver graft to IRI is subjectively assessed using the donor's age, degree of fatty change, and length of ischemia time. However, this information is inaccurate to predict outcomes, and any organs in doubt might be preemptively declined. If the degree of injury can be measured objectively before reperfusion, a new approach can be developed for accurately selecting acceptable organs.

### Machine Perfusion for Organ Preservation: Its Promise and Limitations

Static cold storage (SCS) has been the standard organ preservation technique for the past five decades because of its simplicity and cost effectiveness. In SCS, the organ is stored in cold solution after removal from the donor until implantation. In the past decade, technical refinement has improved the function of machine perfusion, and there has been a resurgence in interest in the use of machine perfusion for marginal grafts to circumvent the limitations of SCS. In 2009, a randomized controlled trial of 336 consecutive deceased kidney donors showed superior 1-year graft survival for kidneys treated with machine perfusion compared with contralateral kidneys preserved in SCS. The success of machine perfusion in kidney transplantation has encouraged the expansion of the concept. In 2018, a randomized trial with 220 LTs demonstrated that normothermic machine perfusion preservation was associated with a 50% lower organ discard rate compared SCS.

Machine perfusion is expected to offer an opportunity to review the viability of organs, and various biomarkers are under investigation. In normothermic machine perfusion for LT, viability criteria for predicting graft survival have been suggested based on a prerequisite condition of either a low perfusate lactate level or evidence of bile production. Importantly, the criteria have never been externally validated. Currently, the only way of proving the benefit of machine perfusion is by demonstrating prolonged graft survival, and the lack of a reliable biomarker is a major barrier in expanding the clinical utility of machine perfusion. An ideal biomarker should reflect the real-time status of tissue damage, condition of the entire organ, quantifiable degree of injury, and effect of treatment in resuscitation. Furthermore, the result should be reproducible and immediately available. It is suggested that investigation of the bile metabolism in hepatic IRI may provide an opportunity for identifying the ideal biomarker and treatment target for the reasons outlined below.

Cholestasis: Reflection of Early Graft Function after LT

As the transplant community continues to broaden the utilization of marginal liver grafts, it has been important to have a valid definition of poor graft function after LT for use in studies for which one intends to correlate biomarkers or genomic profiles with a high probability of graft failure from IRI. As such, early allograft dysfunction has been defined by the presence of one or more of the following variables based on blood tests: (1) cholestasis, (2) increased serum liver enzyme (aminotransferase) levels, or (3) coagulopathy. Primary non-function is fatal and the severest form of early allograft dysfunction, requiring immediate re-transplantation. Primary non-function in LT can be diagnosed via the presence of two of the following features: (1) cholestasis, (2) increased serum aminotransferase level, (3) coagulopathy, and (4) acidosis.

Among the variables, cholestasis is a pathognomonic feature of general hepatic injury. Assessment of cholestasis by measuring serum bilirubin levels has been used to monitor the graft function after LT. Furthermore, visualization of bile secretion after LT has been known to be an excellent prognostic factor. Likewise, the amount of bile production during machine perfusion also has been suggested as a potential biomarker of hepatic viability. However, a recent clinical study suggested that the amount of bile production per se does not predict the outcome. Although the signs of cholestasis (jaundice or hyperbilirubinemia) can only be observed after reperfusion, the underlying condition (i.e., the detrimental cellular activity of bile formation) is presumed to present before reperfusion.

### Mechanism of Bile Formation

Bile is mostly water (95%), and the most prevalent organic solutes in bile are bile salts (3-45 mmol/L) and bilirubin (1-2 mmol/L). Hepatocytes produce bile, and they are highly polarized cells. The basolateral membrane of a hepatocyte occupies 85% of the cell surface, which faces the blood sinusoids. A small portion of the hepatocyte surface (10-15%) is the canalicular membrane, which consists of a wall of the bile canaliculus. Bile formation is the process of the uptake of bile salts and other organic solutes from the basolateral membrane and the excretion at the canalicular membrane; this occurs through the function of proteins that are collectively known as hepatocyte membrane transporters (HMTs).

Among the major subtypes of HMTs, organic anion-transporting polypeptide 1 (OATP1) on the basolateral membrane and multidrug resistance-associated protein 2 (MRP2) on the canalicular membrane are responsible for bilirubin metabolism. Sodium-taurocholate cotransporter (NTCP) on the basolateral membrane and the bile salt export pump (BSEP) mediate transportation of bile acids via hepatocytes. Canalicular bile salt concentrations are more than 1,000-fold higher, necessitating active transport across the canalicular membrane using adenosine triphosphate (ATP). As such, transport across the canalicular membrane is the rate-limiting step in overall hepatocellular bile salt excretion. The process is mediated by MRP2 and BSEP. BSEP secretes monovalent bile salts, such as taurocholate, whereas MRP2 mediates the export of divalent organic anions, such as bilirubin glucuronides and bile salt conjugates. Meanwhile, multidrug resistance-associated protein 3 (MRP3) on the basolateral membrane is responsible for diverting substrates, such as bilirubin and bile acids, by extruding those solutes back into the sinusoids to prevent overload in the hepatocytes. The bile formation is mainly controlled by transcriptional and posttranslational changes of HMT expression.

### Two Ways of Regulating HMTs: Synthesis and Localization

A transcription factor is a protein that binds to DNA to regulate a certain group of gene expressions. When a transcription factor interacts with its binding site on a DNA segment, the response element, the rate of transcription is promoted to increase mRNA abundance. A few transcription factors are known to be involved in HMT synthesis; these include signal transducer and activator of transcription 5 (STAT5), liver receptor homolog 1 (LRH-1), and farnesoid X receptor (FXR). Above all, nuclear factor erythroid 2—related factor 2 (NRF2) may be the most relevant transcription factor in this project because the antioxidant response element (ARE) is the response element for NRF2 that is responsible for the activation of more than 250 genes during oxidative stress. Interestingly, HMT genes like Mrp2, Mrp3, and Oatp1 are also known to be regulated by NRF2. Therefore, HMT expression is transcriptionally regulated, and during IRI with oxidative stress, it is presumed to be controlled by NRF2 (FIG. 9).

The activity of HMT is also regulated by dynamic localization via a vesicle-based trafficking system. Once HMTs are synthesized from the rough endoplasmic reticulum, they are delivered to a submembrane compartment, followed by exocytic targeting to the basolateral or canalicular membrane. The process is reversible, and HMTs exist in a recycling pool for rapid mobilization and insertion between the submembrane vesicle and plasma membrane. Various cholestatic insults lead to internalization of HMT proteins into the submembrane compartment. Especially, oxidative stress induces internalization of MRP2 and BSEP mediated by cPKC and Ca²⁺/NO/nPKC signaling. As such, the function of HMT during hepatic IRI can be regulated by transcriptional (e.g., NRF2-mediated HMT expression changes) and posttranslational mechanisms (e.g., dynamic translocation of HMTs; FIG. 9).

### Hypothesis: The HMT Function Determines Bile Metabolism in Hepatic IRI

Alteration of the bile metabolism during hepatic ischemia could be measured by the function of HMTs, and the altered molecular profiles may be detected during organ preservation before LT. The better understanding of the pathophysiology will provide not only an opportunity to identify a surrogate marker of organ damage but also a potential therapeutic target for preventing hepatic IRI during organ preservation. Approaches to test the hypothesis are described below.

### II. Approaches

We will elucidate the molecular events of HMT dysfunction in hepatic IRI for developing a pharmaceutical intervention and monitoring tool for the function of HMTs. We will implement the strategy outlined below to reach the goal.

### (1) Elucidating Molecular Events of HMT Dysfunction in Hepatic IRI

### INTRODUCTION

Seven major HMTs will be important for developing our data. OATP1B1, MRP2, NTCP, and BSEP are the main transporters for bilirubin and bile acids. MRP3 is responsible for the diversion of substrates back into the sinusoidal blood when hepatocytes are oversaturated. Cl—/HCO₃ exchanger 2 (AE2) is responsible for the secretion of bicarbonate from hepatocytes. Finally, multidrug-resistance protein 2 (MDR2; MDR3 in humans) is responsible for transportation of fluorescent probes that can be used as a functional assessment tool. We have been investigating the biological response of the HMTs to hepatic IRI using the abovementioned transporters as described below.

Preliminary Data 1A: Brief ischemia can increase transcriptional activity of HMTs

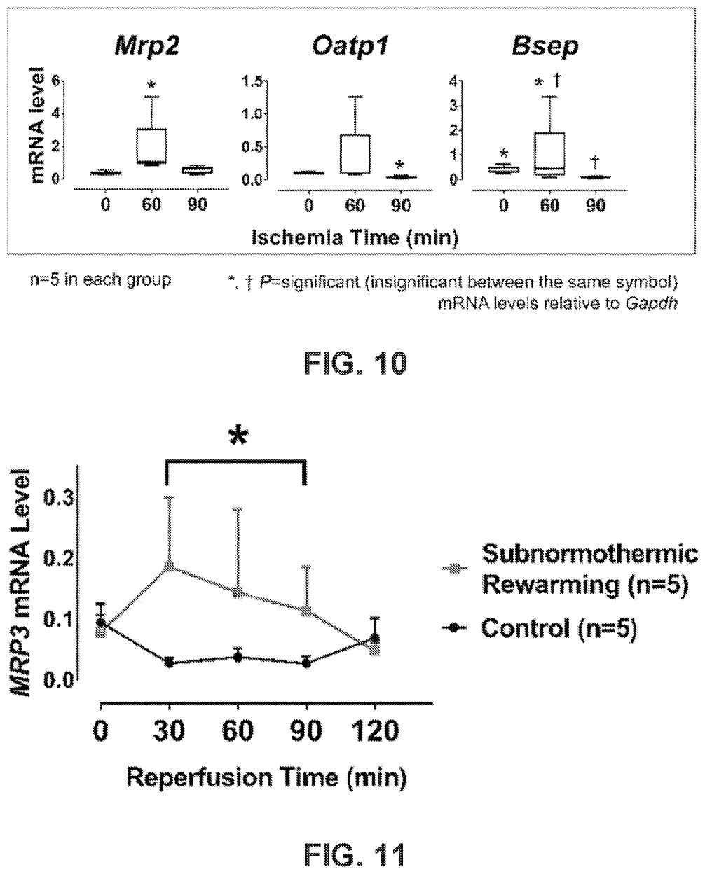

An objective way of controlling organ damage in IRI is by adjusting the ischemia time. In a cardiac IRI model using rats, mRNA expression of a transcription factor (activating transcription factor 3 (Atf3)) increased with hypoxia time for 60 min, then declined. It has also been determined that normal human livers can tolerate 60 min of continuous normothermic ischemia; ischemia beyond 60 min is considered extreme. In animal studies, 90 min of hepatic ischemia has been established as a model for severe IRI. We also previously demonstrated that post-reperfusion mitochondrial redox states of in rat livers were significantly suppressed after 90 min of ischemia compared with those after 60 min of ischemia. As such, it is plausible that molecular profiles of liver tissues demonstrate different patterns in terms of their tolerability to ischemic injury over a boundary between 60 and 90 min. These previous studies and our preliminary data establish a scientific premise supporting the notion that the transcriptional profiles in liver tissues depend on the ischemia duration. We performed a pilot study using rats to observe the effects of the duration of ischemia in liver tissue. The model was achieved by establishing a temporary occlusion of the vascular pedicle to the median and lateral lobes of the liver (70% of the liver volume) using a clamp. We discovered that the transcription activities of HMTs in liver tissue are increased after brief ischemia (60 min). In contrast, after prolonged ischemia (90 min), mRNA expression of HMTs was unchanged or decreased below the baseline level (FIG. 10). The results may imply that hepatocytes increase the transcriptional activity of HMTs during ischemia and the effect disappears when the ischemia time is beyond a certain limitation.

### Preliminary Data 1B: Lower Temperature During Reperfusion Increases HMT Expression in an Ex Vivo Pig Liver Perfusion Model

In clinical LT, the graft temperature suddenly increases from <4° C. in the cold preservation solution to the level of body temperature at the time of implantation. It has been suggested that gradual rewarming during the initial period of reperfusion may be beneficial for hepatic graft function. We investigated the effect of subnormothermic rewarming on the HMT mRNA expressions using a pig liver ex vivo reperfusion model. In the control group, reperfusion was performed using oxygenated whole blood at normothermic temperature (38° C.). In the subnormothermic rewarming group, a lower temperature setting (28° C.) was provided during the first 30 min and the temperature was increased to 38° C. afterward. The subnormothermic rewarming group showed significantly higher mRNA levels of MRP3 for 60 min more (90 min post-reperfusion), and then the effect disappeared (FIG. 11, *P=0.0385, two-way repeated measures analysis of variance (ANOVA)). This result suggests that a temperature change during reperfusion may affect HMT expression levels. It also suggests that the pig model can provide serial tissue samples to investigate the trends of HMT function during hepatic IRI.

### Preliminary Data 1C: Transcriptional Activity of HMTs During Hepatic Ischemia Is NRF2 Dependent

We hypothesized that NRF2 could be an upstream regulator of HMTs in hepatic IRI for the following reasons: First, NRF2 is a regulatory factor of Oatp1, Mrp2, Mrp3, and Bsep in various conditions, such as exposure to acetaminophen or butylated hydroxyanisole. Second, in a rat model, hepatic arterial deprivation was accompanied by a marked reduction in the mRNA levels of Ntcp, Bsep, and Mrp2 after 24 h. Furthermore, it has been reported that mRNA expressions of Ntcp, Oatp1, Bsep, and Mrp2 are decreased after hepatic IRI in a rat model. The change of transcriptional activities in BTs with hepatic IRI implies the involvement of an antioxidant transcriptional factor, such as NRF2. Third, it has been reported that disruption of hepatic glutathione synthesis, which makes the liver vulnerable to oxidative stress, induced expression of Mrp2, Mrp3, and Mrp4. Moreover, NRF2 activation reproduced the effect on BT expression.

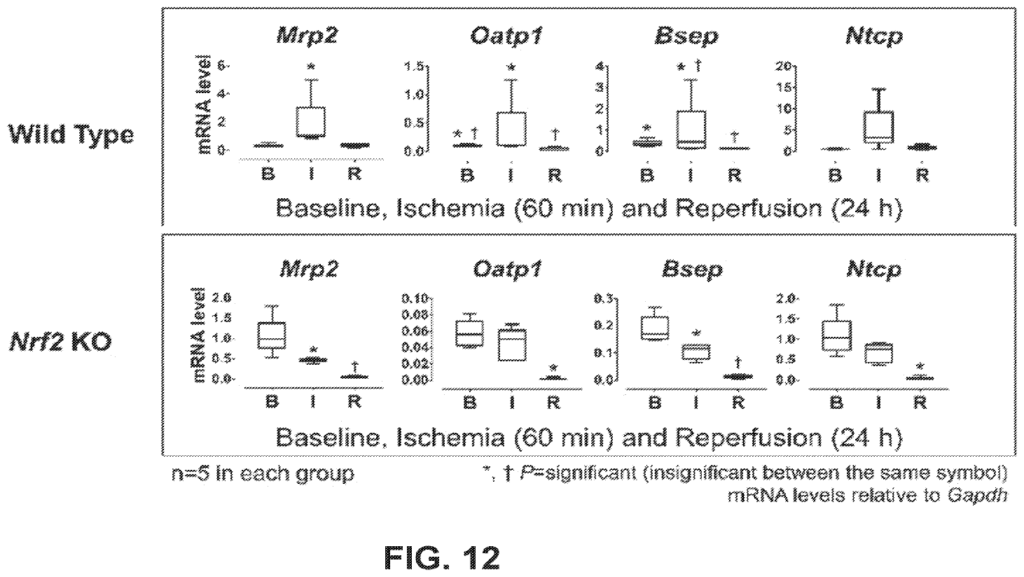

We observed the effect of Nrf2 knockout status in hepatic IRI. Hepatic ischemia for 60 min or the same degree of ischemia followed by 24 h of reperfusion was applied in wild-type and Nrf2 knockout rats. In wild-type rats, 60 min of ischemia increased the mRNA expression of HMTs; it returned to the level of baseline after reperfusion. In Nrf2 knockout rats, the expression did not increase after ischemia; after reperfusion, it decreased to below the baseline level (FIG. 12). Therefore, the transcriptional activities of HMTs during hepatic IRI may depend on the presence of NRF2.

### Preliminary Data 1D: Hepatic IRI Affects Dynamic Localization of HMTs

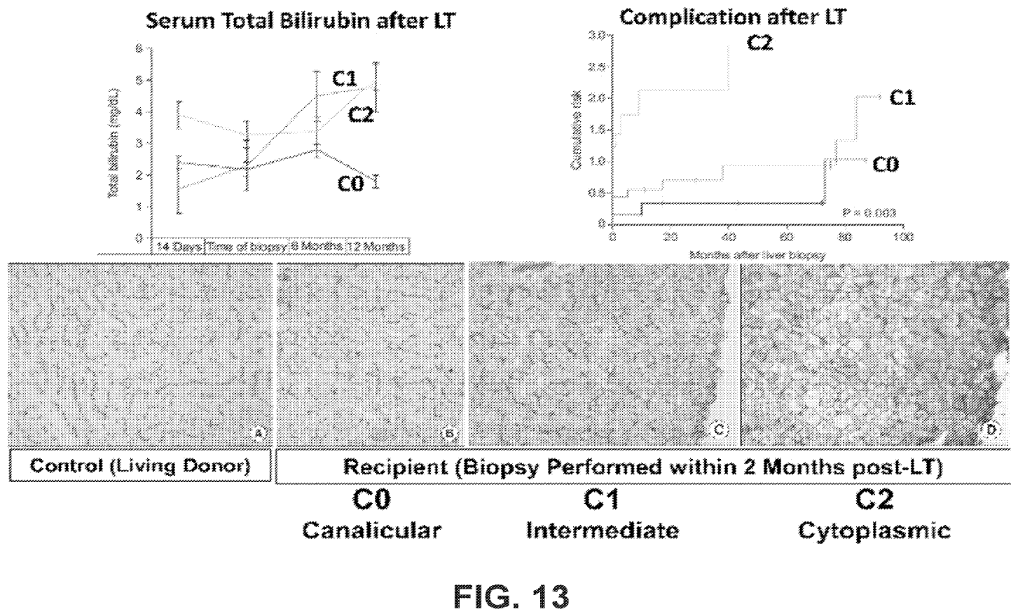

The HMT activity is known to be regulated by dynamic localization of the protein on the hepatocyte cell membrane. The localization is regulated by the vesicle-based trafficking system. To observe the significance of HMT translocation in LT, we analyzed human biopsy samples obtained within 2 months after LT (FIG. 13). Depending on the patterns of immunohistochemistry staining for MRP2, we categorized the cases into three groups: C0 is canalicular, C1 is intermediate, and C2 is cytoplasmic. The C0 (canalicular staining) group showed a lower serum bilirubin level at 1 year after LT, and the C2 (cytosolic staining) group showed a higher incidence of post-transplant complications.

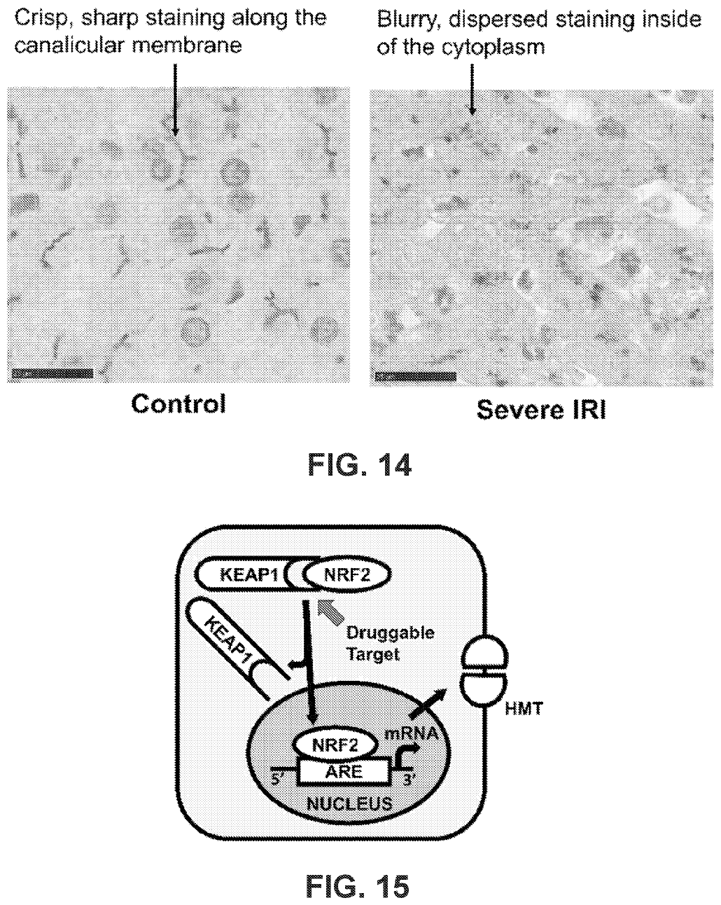

We observed the MRP2 staining pattern in the rat model (FIG. 14). In a physiologic condition, the MRP2 staining was crisp and sharp on the canalicular membrane. After severe IRI (90 min of hepatic ischemia followed by 24 h of reperfusion), the staining was blurry and dispersed into the cytoplasm. As such, dynamic localization may play a role in the function of HMTs during hepatic IRI.

### Overall Approach to Elucidating Molecular Events of HMT Dysfunction in Hepatic IRI

We will observe the effect of four major factors of IRI, which are as follows: ischemia time, temperature, aging, and hepatic steatosis. The biological response of the four factors in hepatic IRI will be studied using molecular analyses in hepatic tissue samples from rat experiments (partial warm ischemia model and ex vivo liver perfusion system) and pig liver grafts in machine perfusion. It is noteworthy that the microscopic area of necrosis in hepatic IRI is irregular and unpredictable. As such, the expression and staining pattern of HMTs should be evaluated in a carefully selected viable area. To minimize sampling error from necrotic tissue sampling, laser capture microdissection will be utilized for sensitive studies.

Immunohistochemistry and digital imaging analysis will be conducted in addition to analysis of bile output and composition.

### (2) Improving the Function of HMT Using Pharmaceutical Intervention Introduction

NRF2 mediates antioxidant gene transcription after oxidative stress. In physiological conditions, NRF2 is retained in the cytosol by binding to its inhibitor, Kelch-like ECH-associated protein 1 (KEAP1, FIG. 15). The oxidative stimuli dissociate NRF2 from KEAP1 and lead to nuclear translocation of NRF2, resulting in transcriptional activation of ARE-regulated genes. The link between NRF2 and KEAP1 is a known therapeutic target. Bardoxolone methyl (BARD) is one of the derivatives of synthetic triterpenoids, and it has been investigated for the treatment of chronic inflammatory diseases and cancer, including phase 3 clinical trials. The binding of BARD to KEAP1 disrupts its cysteine residues, leading to the release of NRF2. The effect of temporal activation of NRF2 by BARD has also been investigated in a mouse model of brain ischemia. Another study demonstrated that daily gavage of oltipraz, another form of NRF2 activation drug, for 4 days consecutively induced Mrp2, Mrp3, and Mrp4 in the normal mouse liver (without IRI).

Preliminary Data 2: BARD Administration Induces HMT Expressions and improves postreperfusion cholestasis

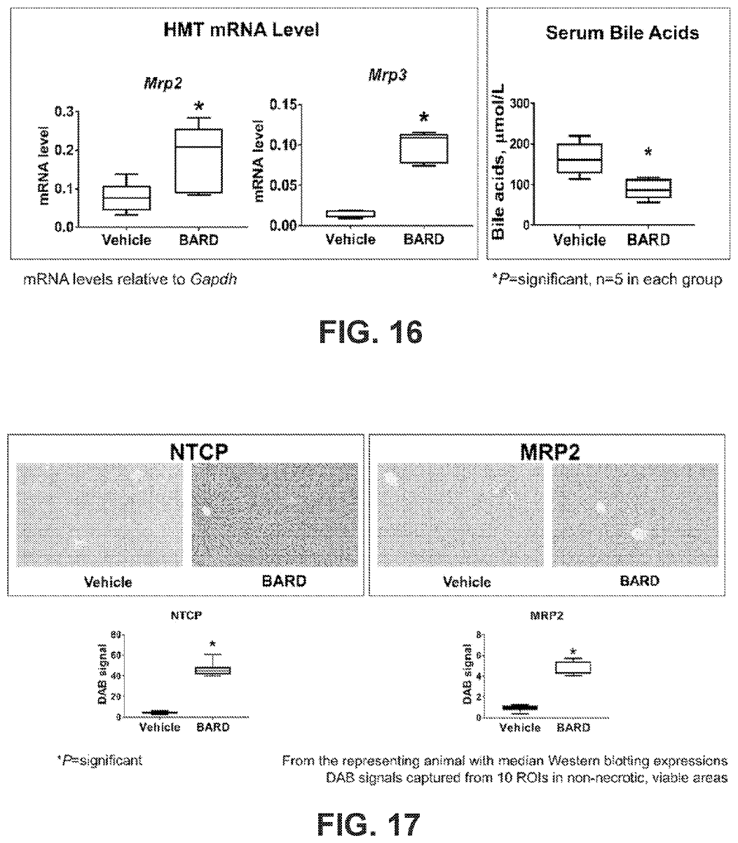

For the utility of NRF2 activator in clinical LT, we tested the effect using the intravenous route, as a single dose protocol in a setting of hepatic IRI. We infused the empty vehicle or BARD solution to the inferior vena cava 5 min before the ischemia and analyzed the liver and blood samples 24 h after reperfusion. The results demonstrated that mRNA expressions of HMTs, such as Mrp2 or Mrp3, were increased by BARD administration. Furthermore, the serum levels of bile acids were significantly decreased in the BARD administration group (FIG. 16). For the first time, we found a clue that it is possible to improve post-reperfusion cholestasis by pharmaceutical intervention at the time of ischemia. Moreover, in immunohistochemistry, 3,3′-diaminobenzidine (DAB) signals for HMTs like NTCP and MRP2 (in a viable area) were significantly increased by BARD administration (FIG. 17).

### Overall Approach to Improving the Function of HMT Using Pharmaceutical Intervention

We will monitor the effect of BARD administration in hepatic tissue samples from rat experiments (ex vivo liver perfusion system), pig liver grafts using a clinical machine pump system (Metra; OrganOx Ltd., Oxford, UK). Molecular signal changes and the effect on bile output and its composition will be monitored. We will also observe the effect of BARD administration in liver grafts with other factors of IRI, such as ischemia time, temperature, aging, and hepatic steatosis. NRF2 and ARE are responsible for the activation of more than 250 genes during oxidative stress that we can analyze based on RNA-sequencing data.

Post-translational regulation mechanisms, such as HMT translocation, are also a therapeutic target. Inhibitors of internalization of HMTs have been reported to be used to control the bile metabolism, namely, cAMP elevation (glucagon or salbutamol), bile acids (taurocholic acid or tauroursodeoxycholic acid), and inhibition of clathrin-dependent endocytosis (chlorpromazine). In summary, we propose two potential therapeutic targets (NRF2 activation and endocytosis inhibition; FIG. 9). Due to the variety of options in details like type, dosage, and the route of the candidate regimen, the development of a monitoring tool for the function of HMT may significantly facilitate developing a new therapeutic approach.

### (3) Developing a Tool to Monitor the Function of HMT in Hepatic IRI

### INTRODUCTION

The function of HMTs can be monitored by using a probe that can be metabolized as a substrate. This approach may provide an opportunity to develop a reproducible method of monitoring the HMT function. Indocyanine green (ICG) is a fluorescent dye that is metabolized by HMTs, such as OATP, NTCP, and MDR3. The clearance of ICG can be monitored by decreasing fluorescence in serial blood samples, and it has been used to predict the tolerance after major liver resection. In addition to ICG, fluorescent probes like sodium fluorescein, rhodamine-123, and DY635 have been investigated to assay HMT function. The fluorescence can also be measured in a live animal using quantitative intravital multiphoton microscopy in different compartments, such as blood, hepatocytes, and biliary canaliculi. The ICG kinetics can also be easily measured by blood clearance in a patient, and it has been reported that the donor's ICG clearance predicts the recipient's cholestasis and the graft survival after LT. The clearance of the HMT probe depends on the blood flow, which is determined by the hemodynamic status of the host. The hemodynamic parameter can be controlled when a liver graft is in machine perfusion, and the ICG clearance may be more accurately measured.

The functional analysis of HMT using the probe has a few advantages over molecular studies. First, the intravital biological condition can be monitored in real time. Second, the entire organ function can be monitored, and it may not be biased by local conditions like necrosis. Third, the degree of function can be reflected by quantitatively measured fluorescence. When the function of HMTs is controlled at the transcriptional and post-translational levels, the functional change could be monitored by intravital imaging and dynamic clearance during the organ preservation.

### Overall Approach to Developing a Tool to Monitor the Function of HMT in Hepatic IRI (Rats)

In this study, male SD rats (250-300 g) will be used, as males are more susceptible to hepatic IRI. Rat models are useful for molecular biological studies of hepatic IRI because the rat is minimally large enough for complex surgical and imaging procedures. Rat models have been used for assessing the function of HMTs by employing fluorescent probes. In normal rats, it takes <30 min for ICG (10 μg/kg) to reach a steady state and <10 min for sodium fluorescein (2 mg/kg). The first step of this project will be the analysis of in vivo kinetics of ICG or fluorescein in hepatic IRI. ICG clearance can be presented as the plasma disappearance rate (ICG_{PDR}, normally over 18%/min) or ICG retention ratio after 15 min (ICG_{R15}, normally less than 10%). The ICG_{R15} can be calculated based on results from blood samples using fluorescence spectrometry at two timepoints, namely, baseline and at 15 min. The probe clearance (ICG_{R15} or fluorescein_{R5}) will be measured at increasing ischemia time (0-90 min) with a fixed reperfusion time (i.e., 24 h). The same experiments will be repeated using older animals or animals with hepatic steatosis induced by a high-fat diet to simulate the effect of IRI on marginal livers. Experiments using quantitative intravital multiphoton microscopy will be performed using the Olympus Fluoview FV1000 MPE multiphoton laser scanning microscope on campus. The region of interest will be carefully selected from the viable area of the liver tissue. This part of experiment will provide the kinetics of probes in the cytosol, bile canaliculi, and sinusoids. The probe kinetics and intravital multiphoton microscopic evaluation will be performed on rat livers in an ex vivo machine perfusion system. The liver will be exposed to up to 60 min of warm ischemia and up to 12 h of cold ischemia for simulating marginal liver grafts in prolonged cold preservation. The effects of Nrf2 knockout status and NRF2 activation on the probe kinetics and intravital microscopy will also be assessed. The correlation of other parameters, such as perfusate liver enzymes, pH, bile output, and bile composition, will be analyzed.

### Overall Approach to Developing a Tool to Monitor the Function of HMT in Hepatic IRI (Pigs)

The function of the clinical machine perfusion system and its monitoring can be further investigated using a pig model that mimics the size and anatomical characteristics of the human liver graft. We conducted a pilot study using the pig ex vivo perfusion model for observing the transcriptional changes during hepatic IRI. The preliminary data demonstrated that a change of specific physiological condition (i.e., temperature) can be reflected by HMT expressions. As such, the pig may provide a reasonably large animal model for investigating the function of HMT in hepatic IRI. The pig livers will be placed in the machine pump system (metra; OrganOx Ltd.) that has been used in clinical studies. A real-time fluorescence sensor will be applied on the tubing of the inflow of the machine pump, and the probe clearance in perfusate will be monitored in liver grafts with different degrees of injury (i.e., different ischemia times). We will also analyze molecular signatures related to the bile metabolism. Among the commercially available probes, ICG and sodium fluorescein are approved for use in human studies, and we will investigate both probes in this project. The effects of NRF2 activation in the pig liver grafts on machine perfusion will also be investigated.

### III. Summary

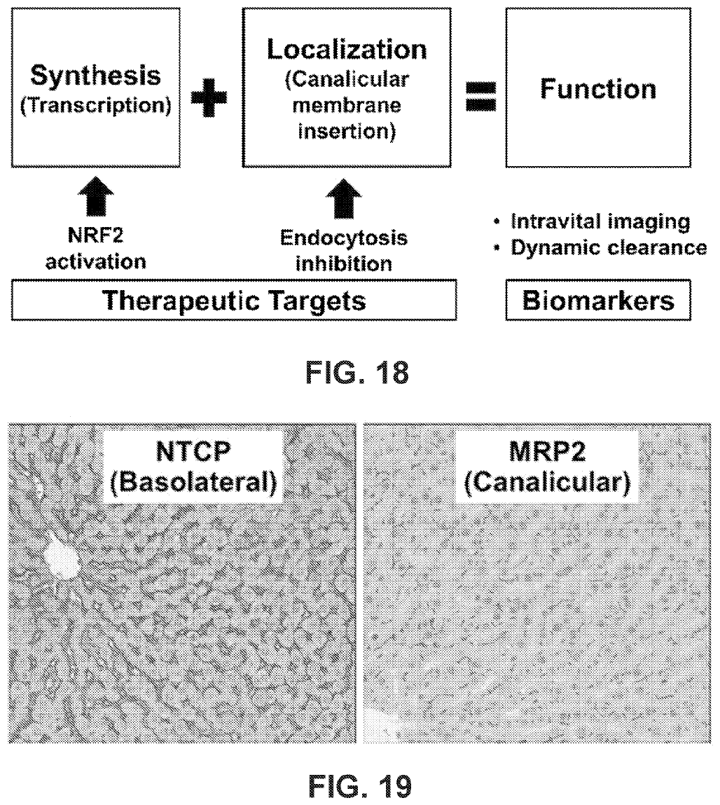

We will examine the role of NRF2 activation and endocytosis inhibition on molecular profiles and functions of HMTs, which can be reflected by intravital imaging and dynamic clearance in experiments using rat and pig livers (FIG. 18). The aim of this effort is to provide fundamental data for a future clinical trial of pharmaceutical intervention of HMT function in the human liver using a machine perfusion system for LT.

The first randomized controlled trial using machine perfusion was published in Nature last year. However, a meaningful monitoring system for assessing the graft quality before LT is still not available. Although we have a novel device to improve the organ function in LT, its application has been limited due to the lack of a monitoring system to prove its benefit. As such, we cannot develop it any further before we add a monitor to it. Disclosed herein are multiple novel concepts in various fields that have never been combined before; expression changes of HMTs, ICG clearance, machine perfusion in LT, and NRF2 activation. The combination of these topics in the present project is presumed to be efficient and exceedingly productive, as each component has already been extensively studied. This project may be unique and novel because a reasonable approach is suggested to build up a novel area of research-monitoring hepatic organ function in a machine perfusion system. If successful, the results of this project will be able to answer one of the most important questions of this era concerning in LT: Can we measure the quality of liver grafts?

We have preliminary data in major topics, as described above, and they will provide a solid starting point for minimizing the chance of failure. As described herein, we will develop them further to combine each topic in a time- and cost-effective way. Furthermore, the diversified approach will prevent the failure of the project. This project is also versatile, as its goal is to find an approach, not to prove the value of a single approach. For example, we will examine the effects of NRF2 inducers (starting with BARD and alternatively employing olanzapine, dimethyl fumarate, CDDO-DFPA, minocycline, sulforaphane, DL-3-n-butylphthalide, ursodiol, resveratrol, or curcumin) or endocytosis inhibitors (glucagon, salbutamol, taurocholic acid, tauroursodeoxycholic acid, and chlorpromazine) in rat and pig livers for measuring ICG (alternatively, fluorescein or rhodamine-123) kinetics via the plasma/perfusate clearance. We may also use intravital multiphoton microscopy for assessing liver grafts with known different degrees of injury (i.e., ischemia times), and we will add additional factors of IRI, such as advanced age, fatty change, or different temperature settings.

In summary, we will develop the project based on the preliminary data to combine multiple components of the topic, with each topic having well-established alternative approaches. We will execute the project using a time- and cost-effective approach, and I will use the best of my experiences to minimize the chance of failure.

### Example 3

### Dynamic Liver Function Test in Machine Perfusion: a Radical Innovation for Organ Assessment in Liver Transplantation

Liver transplantation (LT) is the only hope for end-stage liver disease patients, but many patients die due to the shortage of suitable organs. Unused marginal liver grafts offer a potential resource, but a considerable number of livers have been discarded because of concerns about post-transplant organ failure due to hepatic ischemia/reperfusion injury (IRI). A novel method of organ preservation using machine perfusion has been introduced to improve clinical outcomes, but the use of the machine pump has been limited by the lack of a monitoring system to guide the decision to use marginal liver organs for LT. The overall goal of this project is to improve LT outcomes by developing a system to monitor organ viability. Hepatic IRI is associated with the dysfunction of hepatocyte membrane transporters (HMTs) and results in cholestasis after LT. We hypothesize that the clearance of HMT substrates in machine perfusion may represent the functioning of HMTs and the viability of a liver. The scientific premise is that the degree of liver injury correlates with the biological responses of HMTs in hepatic IRI. The feasibility of our approach is supported by our observation that the degree of IRI affects transcriptional activities and the translocation of HMTs. On this basis, we will examine the clearance of fluorescent substrates of HMTs in three functional compartments (blood, liver tissue, and bile) during hepatic IRI by multiphoton intravital microscopic examination and fluorometric analyses in vivo and ex vivo. The transcription and translocation activities of the HMTs responsible for the metabolism of fluorescent substrates will be quantitatively analyzed using animal models and clinical samples, and the results will be analyzed at each stage of IRI, namely, ischemia, early reperfusion, late reperfusion, and recovery. Therefore, this project aims to develop an objective, real-time monitoring system for liver grafts in machine perfusion based on the unique biological features of HMTs in response to IRI. We will innovate the current practice by elucidating the molecular events of HMT dysfunction in hepatic IRI, allowing the bile metabolism to be monitored during organ preservation for LT. We expect that this project will provide a foundation for clinical trials of a human liver graft monitoring system to test novel methods of organ resuscitation. This work could potentially reduce the preventable deaths of end-stage liver disease patients by improving the success rate and increasing the donor pool in LT.

A significant number of livers from organ donors have been discarded due to concerns about poor quality based on a subjective assessment while patients on the waiting list die without an organ offer for liver transplantation. This project aims to investigate the association between the viability of the liver organ and the functioning of cell membrane proteins that are responsible for bile formation. The dynamic liver function test based on the function of the membrane proteins may serve as a monitoring tool to inform critical decisions for severely ill patients who are waiting for liver transplantation.

### DESCRIPTION

### I. Background

The overall goal of this project is to improve liver transplantation (LT) outcomes by developing a reliable method to predict organ viability. We have been investigating the pathophysiology of cholestasis in hepatic ischemia/reperfusion injury (IRI), and preliminary data led us to identify an opportunity to develop an ex-vivo real-time monitoring system to test liver function, as outlined below.

### I-1. Significance: So Many Livers are Abandoned in the Organ Shortage Era.

End-stage liver disease is a fatal condition. It is the eighth leading cause of death in the United States, and the average life expectancy of patients is as low as two years. The economic burden of liver cirrhosis in the United States is substantial, with annual direct costs exceeding $2 billion and indirect costs exceeding $10 billion. A cure for end-stage liver disease was unavailable until LT was first developed in 1963. Currently, more than 65,000 people in the United States are living with a transplanted liver. However, approximately 12,000 people are still waiting for LT, and over 2,000 patients die annually while on the waiting list. As such, the shortage of donor organs is a critical barrier in the management of patients with end-stage liver disease.

While patients and their families desperately wait for a liver donor to become available, about 1,700 livers from deceased organ donors are discarded each year due to concerns about organ quality.

Unused marginal grafts are a potential resource for addressing organ shortage in LT. The reluctance to use grafts of marginal quality stems from the fear of graft failure, which occurs after LT in approximately 5-8% of cases, even with the high rate of preemptive organ discard. This results in a dilemma in the organ shortage era: avoiding the use of marginal liver grafts increases waiting list mortality, while encouraging it increases post-LT mortality from graft failure (FIG. 8). Currently, the decision is based on subjective data, such as the estimation of organ availability and the severity of the recipient's condition. To develop an objective clinical strategy, it is important to understand the two-stage nature of the pathophysiology underlying IRI.

### I-2. IRI is a Two-stage Phenomenon.

The LT procedure comprises organ procurement from the donor and organ implantation into the recipient. At the time of organ procurement, blood flow in the organ must be interrupted, and tissue ischemia is inevitable. Ongoing ischemic insult to the cells results in the activation of Kupffer cells, the specialized tissue-resident macrophages located in the lining of the liver sinusoids. Six to 24 h after reperfusion of the blood flow in the recipient, neutrophils are recruited into the liver parenchyma due to the inflammatory signal produced by the Kupffer cells. Neutrophils then directly injure hepatocytes via oxidants and proteolytic enzymes, leading to necrotic cell death. Therefore, the degree of tissue damage is determined by the activated Kupffer cells during ischemia, and severe damage is executed by neutrophils during reperfusion.

The final decision on whether to accept a graft should be made before LT, at the time of organ preservation. However, a useful test is not available during organ preservation to determine which choice should be made. As such, the vulnerability of a liver graft to IRI is subjectively assessed using the donor's age, degree of fatty change, and length of ischemia time. However, this information inaccurately predicts outcomes, and any organs in doubt might be preemptively declined. If the degree of injury can be objectively measured before LT, a new approach for selecting acceptable organs can be developed.

### 1-3. Machine Perfusion for Organ Preservation is Promising but has Limitations.

Static cold storage (SCS) has been the standard organ preservation technique for the past five decades because of its simplicity and cost effectiveness. In SCS, the organ is put in cold solution storage (4° C.) after removal from the donor until implantation. In the past decade, technical refinement has improved the function of machine perfusion, leading to a resurgence in interest in its use for marginal grafts to circumvent the limitations of SCS. In 2018, a randomized trial with 220 LTs demonstrated that normothermic (37° C.) machine perfusion preservation was associated with a 50% lower organ discard rate compared to SCS.

When a liver graft is placed in a normothermic machine perfusion system, the liver has already undergone ischemic injury. The graft is then reperfused with perfusate containing leukocyte-filtered blood. Due to the lack of neutrophils in the perfusate, the late phase of reperfusion does not occur while the graft is in machine perfusion. As such, it is plausible that the physiologic function of the liver graft during the early reperfusion phase can be simulated under machine perfusion before LT. In this regard, viability criteria for predicting graft survival have been suggested based on inflammatory markers, anaerobic metabolism indicators, and evidence of bile production. Importantly, none of the criteria have been validated, and the only way to prove the benefits of machine perfusion is to perform the LT based on a so-called gut feeling and demonstrate prolonged graft survival after transplantation. As such, the lack of a reliable biomarker is a major barrier to expanding the clinical utility of machine perfusion. An ideal biomarker should reflect the real-time status of tissue damage, the condition of the entire organ, the quantifiable degree of injury, and the effect of pharmaceutical intervention.

We suggest that investigation of the bile metabolism in hepatic IRI may provide an opportunity to identify the ideal biomarker for the reasons outlined below.

I-4. Cholestasis Reflects Graft Function after LT.

As the transplant community continues to broaden the utilization of marginal liver grafts, it has become important to have a valid definition of poor graft function after LT for use in studies aiming to correlate biomarker profiles with a high probability of graft failure from IRI. As such, early allograft dysfunction has been defined by the presence of one or more of the following variables within seven days after LT based on blood test results: (1) cholestasis, (2) increased serum liver enzyme (aminotransferase) levels, and (3) coagulopathy. Primary non-function is fatal and the severest form of early allograft dysfunction, requiring immediate re-transplantation. Primary non-function in LT can be diagnosed via the presence of two of the following features within seven days: (1) cholestasis, (2) increased serum aminotransferase level, (3) coagulopathy, and (4) acidosis.

Cholestasis is a pathognomonic feature of hepatic injury. Assessment of cholestasis by measuring serum bilirubin levels has been used to monitor graft function during the first week after LT. Furthermore, visualization of bile secretion after LT is an important prognostic factor. Although signs of cholestasis, such as high serum bilirubin levels and jaundice, can only be observed a few days after reperfusion, the underlying condition (i.e., the detrimental cellular activity of bile formation) is presumed to be present before reperfusion.

### 1-5. Hepatocyte Membrane Transporters are Responsible for Bile Formation.

Bile is mostly water (95%), and the most prevalent organic solutes in bile are bile salts (3-45 mmol/L) and bilirubin (1-2 mmol/L). Hepatocytes, which produce bile, are highly polarized cells. The basolateral membrane of a hepatocyte occupies 85% of the cell surface, which faces the blood sinusoids. A small portion of the hepatocyte surface (10-15%) is the canalicular membrane, which consists of a wall of the bile canaliculus. Bile formation is the process of the uptake of organic solutes from the basolateral membrane and their excretion at the canalicular membrane; this occurs through the function of proteins located along each cell membrane that are collectively known as hepatocyte membrane transporters (HMTs). Different kinds of HMTs are located alongside the basolateral membrane and the canalicular membrane due to the polarized function of the hepatocyte (representative markers are shown in FIG. 19). Among the major subtypes of HMTs, organic anion-transporting polypeptides (OATPs) and sodium-taurocholate cotransporter (NTCP) are located on the basolateral membrane, and they are responsible for uptake of organic solutes such as bilirubin and bile acids from blood; multidrug resistance-associated protein 2 (MRP2) and bile salt export pump (BSEP) on the canalicular membrane are responsible for excretion into bile. Canalicular bile salt concentrations are more than 1,000-fold higher than those in hepatocytes, necessitating active transport across the canalicular membrane using adenosine triphosphate (ATP). Transport across this membrane, mediated by MRP2 and BSEP, is considered the rate-limiting step in overall hepatocellular bile salt excretion. Meanwhile, multidrug resistance-associated protein 3 (MRP3) on the basolateral membrane is responsible for diverting substrates, such as bilirubin and bile acids, by extruding those solutes back into the sinusoids to prevent overload in the hepatocytes. Bile formation is a highly regulated process, and it responds immediately to stressful stimuli. Thus the key question of this project is whether the biological response of bile metabolism in hepatic IRI corresponds to the degree of injury. We developed preliminary data to demonstrate the validity of the question.

### II. Preliminary Studies

### II-1 Introduction: Machine Perfusion as a Tool to Assess Liver Graft Viability

The monitoring methods for liver grafts in machine perfusion described in the literature are mainly based on perfusate analysis of biomolecules, such as the release of hepatocellular enzymes (e.g., aminotransferases and lactate dehydrogenase) or indicators of anaerobic metabolism (e.g., lactate clearance). Unfortunately, no guideline could be established based on those parameters due to their weak association with LT outcomes. Furthermore, when the normothermic machine perfusion system was first introduced, many expected that the amount of bile production from a liver graft could predict LT outcomes. However, the liver grafts in machine perfusion showed a random pattern of bile production with regard to their viability. Moreover, bile production measurement can be confounded by the administration of additives that affect the amount of bile production positively (e.g., bile salts) or negatively (e.g., high dose glucose). Although the amount of bile production per se does not reflect the viability of an organ, it is widely accepted that a liver graft with poor function develops cholestasis after transplantation. As such, biological alteration of the bile production system is presumed to present in the liver graft before signs of cholestasis become evident.

### II-2. Brief Ischemia Increases Transcriptional Activities of HMTs.

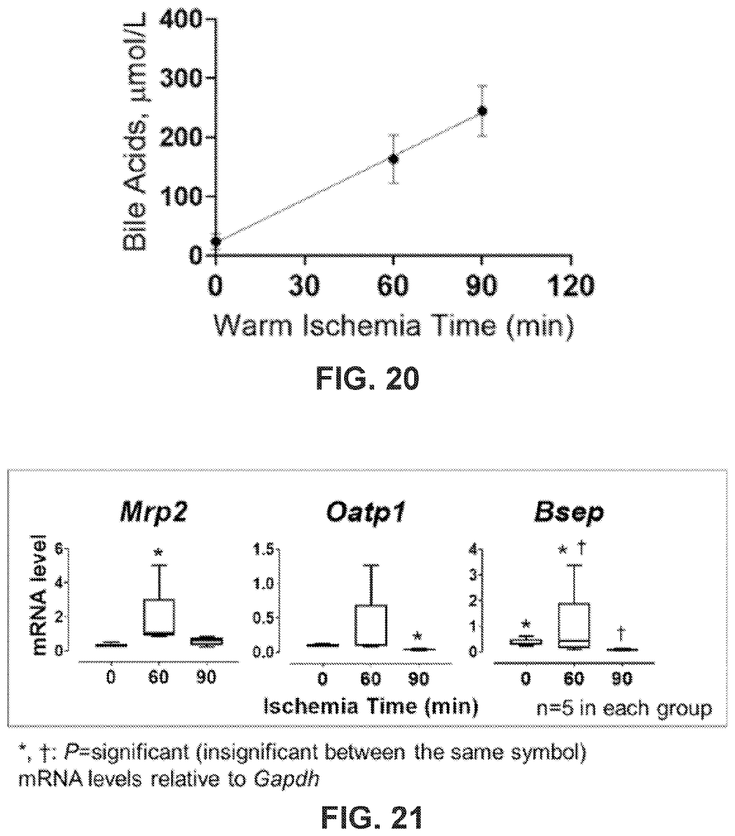

In an experimental setting, organ damage in IRI can be objectively adjusted by the ischemia time. We have observed that the duration of ischemic injury correlates to the degree of cholestasis (e.g., serum levels of bile acids) after reperfusion in a rat model (FIG. 20). As the ischemia time correlates to the degree of liver damage, it is presumed that there is a maximum limit of ischemia time that a normal liver can tolerate. In clinical liver surgery, normal human livers can tolerate 60 min of continuous normothermic ischemia. As such, a surgical procedure that occludes hepatic blood flow (e.g., the Pringle maneuver) and lasts longer than 60 min is considered extreme and has a high risk of hepatic failure. In animal studies, 90 min of hepatic ischemia was established as a model for severe IRI. In a cardiac IRI model using rats, mRNA expression of a transcription factor (activating transcription factor 3 (Atf3)) increased with hypoxia time for 60 min and then declined. We also previously demonstrated that post-reperfusion mitochondrial redox states in rat livers were significantly suppressed after 90 min of ischemia compared with those after 60 min of ischemia.

As such, it is plausible that molecular profiles of liver tissues demonstrate different patterns in terms of their tolerability to ischemic injury over a boundary between 60 and 90 min. This observation establishes a scientific premise supporting the notion that transcriptional profiles in liver tissues depend on the length of ischemia time. We discovered that the transcription activities of HMTs in liver tissue increased at 60 min of ischemia. In contrast, at 90 min, the mRNA expression of HMTs was unchanged or decreased below the baseline level (FIG. 21). These results imply that the mRNA abundance of HMTs in the liver increases during ischemia and that the effect disappears when the ischemia time is beyond a critical limit. This observation may be a clue that the maximum tolerable ischemia time of a specific liver tissue can be measured by measuring the mRNA levels of HMTs. This discovery is interesting because cutoff values of a surrogate marker for organ acceptance in LT have been set subjectively.

### II-3. Transcription of HMTs is a Sensitive Indicator of Physiologic Response During the Early Phase of Reperfusion.

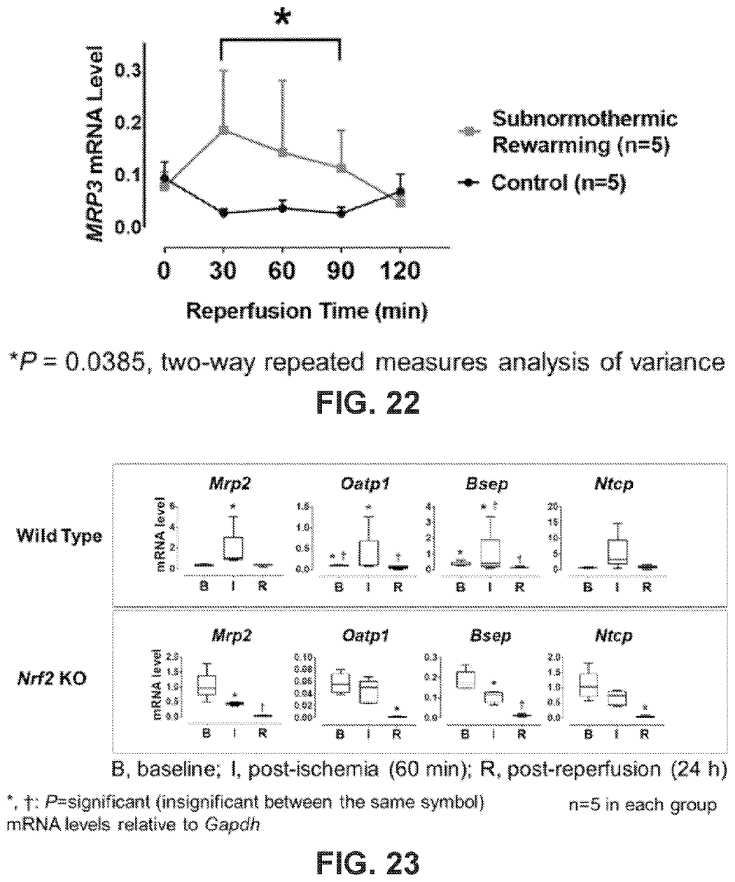

It has been suggested that gradual rewarming during the initial period of reperfusion may be beneficial for hepatic graft function. We investigated whether the effect of subnormothermic rewarming can be reflected by the transcription of HMTs using a pig liver ex vivo reperfusion model. In the control group, reperfusion was performed using oxygenated whole blood at the normothermic temperature (38° C.). In the subnormothermic rewarming group, a lower temperature setting (28° C.) was used during the first 30 min, and the temperature was increased to 38° C. afterward. The subnormothermic rewarming group showed significantly higher mRNA levels of MRP3 during the early reperfusion phase (FIG. 22). The results suggest a regulation mechanism through which the tissue condition in the liver during IRI can immediately be reflected by the mRNA levels of HMTs.

II-4. NRF2 Regulates the Transcriptional Activity of HMTs during Hepatic Ischemia.

In 2019, the Nobel Prize was awarded to researchers who elucidated how cells respond to oxygen availability; a hypoxic environment changes the chemical structure of transcription factors responsible for the cellular response to ischemia. A transcription factor is a protein that binds to DNA to regulate a certain group of gene expressions. When a transcription factor interacts with its binding site on a DNA segment (i.e., the response element), the rate of transcription is promoted to increase mRNA abundance. In this regard, the action of transcription factors may be the first step in reactive changes in bile metabolism. HMT genes are known to be targets of various transcription factors, such as signal transducer and activator of transcription 5 (STAT5), liver receptor homolog 1 (LRH-1), hypoxia-inducible factor 1 (HIF-1), farnesoid X receptor (FXR), and nuclear factor erythroid 2-related factor 2 (NRF2).

NRF2 may be the most relevant transcription factor in hepatic IRI because the antioxidant response element (ARE), which is the response element for NRF2, is responsible for the activation of more than 250 genes during oxidative stress. We observed the effect of Nrf2 knockout status in hepatic IRI. Hepatic ischemia for 60 min or the same length of ischemia followed by 24 h of reperfusion was applied in wild-type and Nrf2 knockout rats. In wild-type rats, 60 min of ischemia increased the mRNA expression of HMTs; it returned to the baseline level after reperfusion. In Nrf2 knockout rats, the expression did not increase after ischemia; after reperfusion, it decreased to below the baseline level (FIG. 23). These observations suggest that the trigger of HMT gene expression in IRI may be dependent on NRF2.

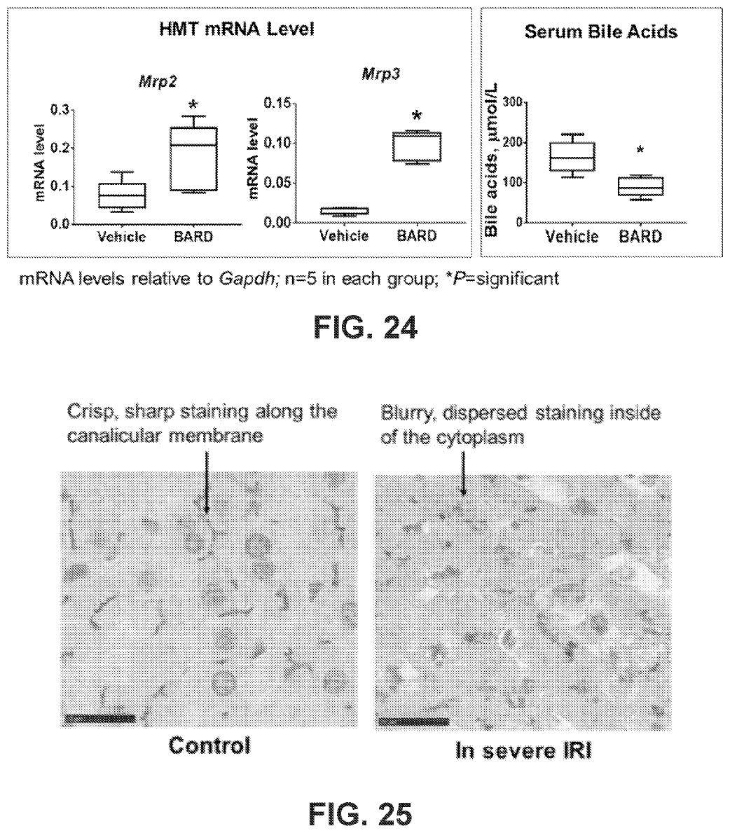

Under physiological conditions, NRF2 is sequestered by Kelch-like ECH-associated protein 1 (KEAP1) in the cytosol. Oxidative stimuli can induce dissociation of NRF2 from KEAP1 and its subsequent nuclear translocation, triggering transcription of ARE-regulated genes. The physical interaction of NRF2 and KEAP1 is a known target for the design of a therapeutic strategy. For example, bardoxolone methyl (BARD), a derivative of synthetic triterpenoids that binds to KEAP1, inhibits KEAP1-NRF2 interaction and induces the release of NRF2 from the complex, eventually leading to the activation of NRF2 as a transcription factor. We tested the effect using the intravenous route for BARD administration as a single dose protocol. We infused the empty vehicle or BARD solution into the inferior vena cava 5 min before the ischemia and analyzed the liver and blood samples 24 h after reperfusion. The results demonstrated that mRNA expressions of HMTs, such as Mrp2 or Mrp3, were increased by BARD administration. Furthermore, the serum levels of bile acids were significantly decreased in the BARD administration group (FIG. 24). Accordingly, we speculate that HMT gene expression indicates that the degree of hepatic IRI and NRF2 may play a role in the mechanism.

Of note, the mRNA level changes did not correlate with those of the protein expression levels in the model. This lack of correlation has been described in the literature, and it is thought to result from the multilayered pre- and posttranscriptional regulation of HMTs. As such, the lack of changes in corresponding HMT protein levels suggests another regulatory mechanism.

### II-5. Dynamic Localization is Another HMT Regulation Mechanism.

Half-lives of most membrane proteins are longer than 24 h; the half-life of MRP2 is 22 to 36 h, and the half-life of BSEP is 4 to 6 days. As such, it may take a few days to observe the effect of the transcriptional regulation of HMTs. However, ischemia is an acute condition to which cells are required to respond immediately. In this regard, another regulation mechanism for the function of HMTs has been discovered: dynamic localization via a vesicle-based trafficking system.

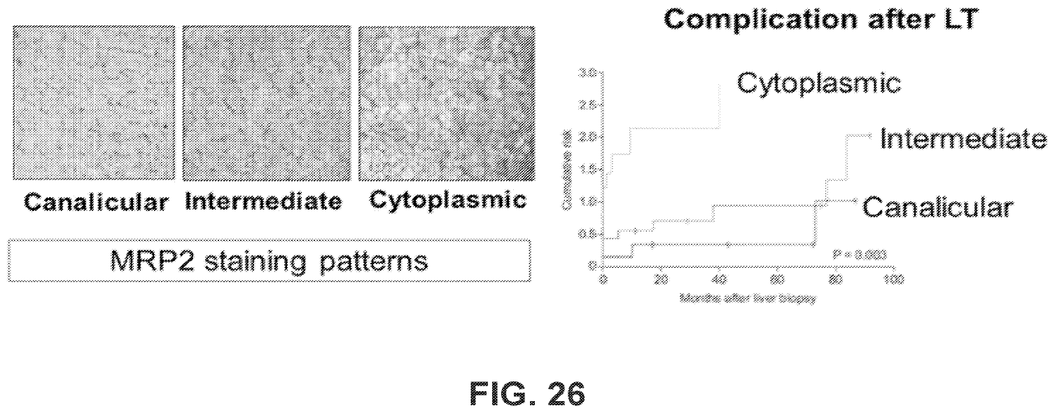

HMTs exist in a recycling pool for rapid mobilization and insertion between the submembrane vesicle and the plasma membrane. This has been suggested as a mechanism of cholestasis in oxidative stress because a prooxidant compound induces the internalization of HMTs, and it can be reverted by glutathione, an antioxidant. Moreover, the alteration of bile flow in hepatic IRI has been shown to coincide with MRP2 translocation. We observed IRI-induced translocation of canalicular HMTs (e.g., cytosolic staining pattern) in our rat model. In a physiologic condition, the MRP2 staining was crisp and sharp on the canalicular membrane. After severe IRI (90 min of hepatic ischemia followed by 24 h of reperfusion), the staining was displaced into the cytoplasm (FIG. 25).

We also analyzed clinical biopsy samples obtained within two months after LT to observe the clinical significance of HMT translocation. Depending on the patterns of immunohistochemistry staining for MRP2, the cytoplasmic staining group showed a higher incidence of post-transplant complications (FIG. 26). As such, dynamic localization may play a clinically significant role in the function of HMTs in hepatic IRI.

### II-6. Dynamic Localization can be Quantified by Colocalization Analyses.

If the degree of HMT protein translocation along the cell membrane can be measured, it may reflect the degree of liver injury and recovery. A method to measure the subcellular localization of HMTs reported in the literature entails manually measuring the distance of fluorescence from the center of the cell membrane (estimated by tight junction markers, such as ZO-1 or occludin) along a line placed perpendicular to the membranes of adjacent hepatocytes. However, results based on this method only represent the status of the point where it is measured, and this method may not be appropriate for a model with heterogeneous injury patterns inside the liver, such as in IRI.

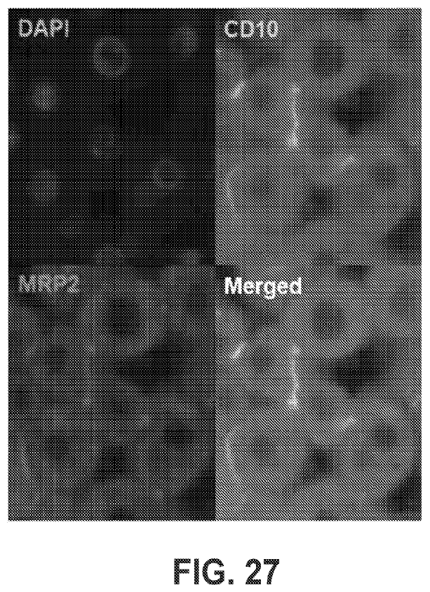

The degree of translocation of a target HMT can be measured using quantitative colocalization analysis (e.g., Mander's correlation coefficient analysis). A few immunostaining markers have shown canalicular membrane staining (e.g., CD10, p-CEA, and CD13) and basolateral membrane staining (e.g., Na⁺/K⁺-ATPase and CD59). We investigated an immunofluorescence staining method for colocalization of a canalicular HMT (MRP2) and a canalicular membrane marker (CD10), demonstrating colocalization in a normal liver (FIG. 27). When an optimized protocol is available, the cut surface of an entire lobe of a rat liver can be scanned for digital analyses to provide a non-biased, quantitative estimation of HMT translocation.

As such, it is plausible that the biological response of bile metabolism in hepatic IRI may reflect the degree of injury. Based on these findings, we hypothesize that transcriptional and post-transcriptional changes of HMTs during the early reperfusion phase of IRI represent the degree of cholestasis at its final phase. We further hypothesize that the clearance of HMT substrates by hepatocytes may represent the viability of a liver in machine perfusion. The overall hypothesis can be tested by fluorometric analyses, as described below.

### III. Approaches

### III-1. HMT Function Will be Measured by Fluorometric Analysis.

Measuring clearance rates of extrinsically administered markers metabolized by HMTs can be an objective method for quantifying their function. Indocyanine green (ICG) is a fluorescent dye that has been used to assess liver function in the field of liver surgery for more than 60 years. ICG is metabolized exclusively by the liver, and ICG clearance can be monitored by decreasing fluorescence in serial blood samples after intravenous administration; clearance at 15 min is the most commonly used measurement. ICG retention values above 10-15% at 15 min are considered abnormal and are used as a cutoff to identify patients at risk for liver failure following major liver resection surgeries. ICG kinetics can also be measured by a portable, finger-probe-based device that measures ICG plasma disappearance rates, and donor ICG kinetics measured before organ procurement has been shown to predict the recipient's cholestasis and graft survival after LT. Recently, ICG clearance was found to be the function of specific HMTs, such as OATP1B1/3, NTCP, and multidrug resistance protein 3 (MDR3). As such, it is plausible that ICG clearance in machine perfusion reflects the function of HMTs. Measurement in machine perfusion is supposed to be more accurate than measurement from a patient because hemodynamic parameters can be strictly controlled in a machine perfusion system. Moreover, the kinetics can be measured not only from perfusate but also from the liver surface and bile in machine perfusion, which is impossible in a patient.

### III-2. Multiphoton Intravital Fluorometric Analyses Will Provide Insights in Cell-Level Pathophysiology.

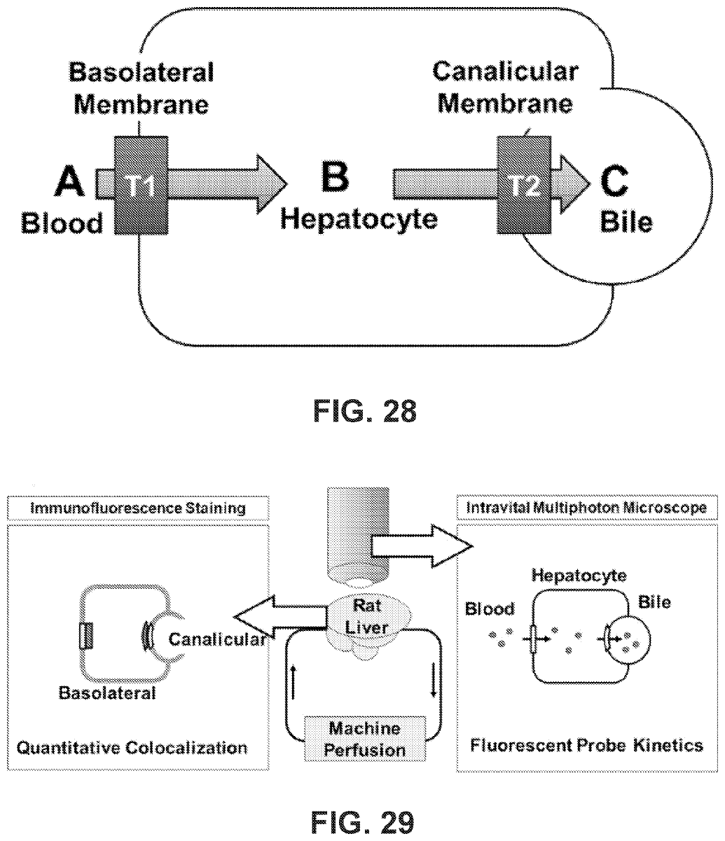

The measurement of fluorescence in blood, the liver surface, and bile in a machine perfusion system may represent values measured at the cell level from sinusoids, hepatocytes, and biliary canaliculi, respectively. In HMT substrate metabolism kinetics, three compartments are divided by two types of cell membrane (FIG. 28). OATPs and NTCP are responsible for uptake of ICG into hepatocytes, and MDR3 (MDR2 in rats) excretes ICG into bile. Since the timing and degree of response to IRI can vary among HMT subtypes, differentiating the functions of uptake and excretion can be an important point. Intravital multiphoton fluorescence excitation microscopy provides the spatial and temporal resolution necessary to characterize hepatic transport at the hepatocyte, sinusoid, and biliary canaliculi levels in vivo and thus to identify the bile metabolism mechanism (FIG. 29). Interestingly, it has been reported that uptake of ICG exceeds excretion, resulting in a hepatocellular accumulation of ICG even in healthy animals. ICG is a useful indicator for a blood sample-based study, but its role in a study of HMT biology may be limited due to the slow clearance rate from the hepatocyte into bile.

As an alternative indicator, fluorescein has been investigated because it passes efficiently through the basolateral and canalicular membranes, and it is approved for use in human studies. The equilibrium of fluorescence intensity in hepatocytes and bile can be achieved within 5 min of dye injection. Sodium fluorescein is taken up by OATP1B1/3 (OATP1B2 in rats) and excreted by MRP2. The faster clearance of sodium fluorescein from hepatocytes may be related to the efficient function of MRP2 compared to that of MDR2. The rapid clearance of sodium fluorescein could make it possible to determine the site of HMT dysfunction (Table 1). This model provides an opportunity to compare the effects of IRI on HMT translocation (e.g., quantitative colocalization of HMTs) and the kinetics of HMT substrate transports (e.g., clearance of fluorescence among compartments).

TABLE 1
Fluorescence kinetics after injection of
HMT substrate and their interpretation

| | Blood | Hepatocytes | Bile | Interpretation |
|---|---|---|---|---|
| Fluorescence | Rapid | Rapid | Rapid | No HMT |
| after | decrease | increase | increase | dysfunction |
| injection | Slow | Slow | Slow | Basolateral HMT |
| | decrease | increase | increase | dysfunction |
| | Rapid | Rapid | Slow | Canalicular HMT |
| | decrease | increase | increase | dysfunction |

If we prove the hypothesis that the clearance of HMT substrates represents the viability of a liver organ, we will be able to develop an objective real-time monitoring system for liver grafts in normothermic machine perfusion based on the unique biological features of HMTs in response to IRI.

### IV. Study Design

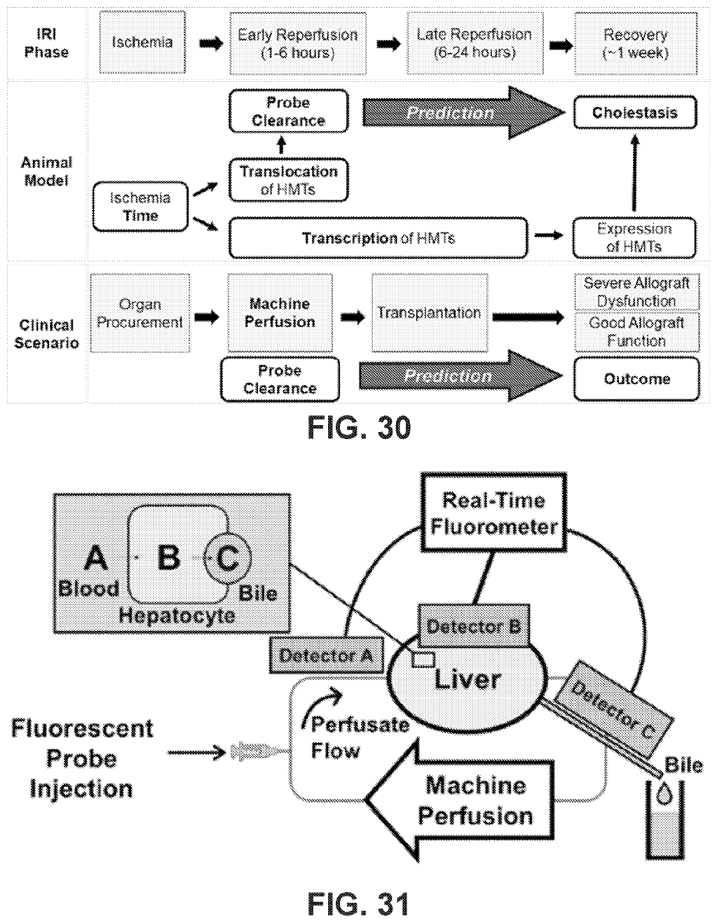

Our study design is based on the sequence of events in the hepatic IRI model and in clinical LT (FIG. 30). During the early phase of IRI (1-6 h), organ damage is limited due to the lack of neutrophil infiltration into the tissue. We hypothesize that the biological response of the liver graft during the early reperfusion phase can predict the degree of damage at the late reperfusion phase (6-24 h), which will, in turn, affect the outcome at the recovery phase (˜1 week). The early reperfusion phase can be simulated by a leukocyte-filtered normothermic machine perfusion system to avoid neutrophil-induced tissue destruction, and this phase can be prolonged until the graft in the perfusion system is implanted in a recipient. The scientific premise of this study is that the degree of hepatic IRI correlates with the biological responses of HMTs. In the experimental setting, the ischemia time can be gradually increased, which will be reflected by the two biological responses of HMTs: translocation and transcription. The effect of translocation can be immediately reflected by the function of HMTs, which can be assessed by fluorometric analysis. The fluorescence of HMT probes can be measured at three sites of the machine perfusion: from perfusate, from the liver surface, and from bile (FIG. 31). This approach will allow monitoring of the quantitative assessment of HMT functions of the entire liver graft in real time. Meanwhile, the effect of transcription on protein expressions will take a few days and occur in a delayed fashion. As such, if the fluorescent probe clearance and the transcription activity are proportional to the degree of ischemic damage, then the probe clearance during the early reperfusion phase should correlate to the expression of HMTs and their function during the recovery phase. We hypothesize that the probe clearance during the early reperfusion phase (either in vivo or ex vivo) could correlate with the degree of cholestasis at the recovery phase in hepatic IRI. An integrative approach will be adopted using the following experimental platforms of rat, porcine, and human liver models to address the multilayered regulation of HMTs in hepatic IRI.

IV-1. How does HMT Biology Respond to Known Factors in Hepatic IRI?(in Vivo Rat Total Hepatic IRI Model)

Although the biological condition of the in vivo hepatic IRI model does not include cold ischemia time as in clinical LT, all IRI stages can be simulated with reproducible data, and hemodynamic parameters in the liver during reperfusion can be naturally optimized. As such, this model can provide fundamental information about expressions of HMTs and kinetics of fluorescent HMT transport probes. The pathophysiology of HMT biology responding to known IRI risk factors, such as old age and steatosis, can also be evaluated. Furthermore, the model allows observation of the effect of targeted medicine or knockout status of relevant genes, such as Nrf2.

Of note, most experimental rodent studies for hepatic IRI have been performed using a partial hepatic ischemia model so blood flow from the intestine can flow through the patent portal vein in the remnant part of the liver.

However, the hepatic lobes spared from ischemia will contribute to hepatic clearance and may bias the results. Moreover, the clearance capacity will be restored by hepatic regeneration of the spared liver lobes within a few days. As such, we will utilize a total hepatic IRI model with a temporary venous shunt (spleno-jugular) during the total hepatic ischemia. The total hepatic IRI model may provide more accurate data regarding the clearance of fluorescent substrates in hepatic IRI.

### IV-2. Can a Machine Perfusion System be a Sandbox Tool to Test HMT Biology?(Ex Vivo Rat Hepatic IRI Model)

HMT biology in the normothermic machine pump system can be evaluated in an ex vivo circuit. The liver graft in this model will allow the collection of bile for further biochemical evaluation. The role of transcription factors can be analyzed based on RNA sequencing data in machine perfusion. This model also provides ideal material for the intravital multiphoton microscopic investigation of HMT substrate kinetics to compare fluorometric results between cell and organ levels. Regulation of HMT translocation can also be tested by cAMP elevation (glucagon or salbutamol), bile acids (taurocholic acid or tauroursodeoxycholic acid), and the inhibition of clathrin-dependent endocytosis (chlorpromazine). The ex vivo environment will also allow motion-free high-quality image capturing (due to a lack of respiratory movement) for the intravital microscopic evaluation.

IV-3. How does HMT Biology Look in Clinical Specimens?(Clinical LT Biopsy Samples)

We will analyze translational research samples stored in the tissue biobank from LT cases at our center. This is not only to address physiologic differences between species but also to observe data from clinical samples in a non-experimental setting. The samples are collected at four time points during the course of LT before donor procurement surgery (baseline), after cold static preservation (post-ischemia), after implantation surgery (early reperfusion), and during planned re-operation for staged bile duct and/or abdominal wall closure (recovery phase). The samples will be analyzed for histologic imaging and next-generation sequencing.

Demographic data, surgical factors, and lab results will also be comparatively analyzed.

### IV-4. Can We Validate the Model in a Machine Perfusion System Used in Clinical Practice?(Ex Vivo Porcine Hepatic IRI Model)

Porcine livers in ex-vivo machine perfusion can provide data regarding the correlation between changes in bile metabolism and ischemia time. The large animal model has two important advantages. First, trends in molecular biology for HMTs can be monitored in each animal by serial sampling, which is impossible in rat livers due to the small size of the organ. Second, the clinical machine perfusion setting can be tested; the same protocol and parameters can be used as clinical machine perfusion for LT. The prototype of a monitoring system can also be designed based on the correlation between fluorometric data and ischemia time.

### IV-5. Do HMT Kinetics in Machine Perfusion Represent Clinical Features?(Ex Vivo Preservation of Discarded Human Livers)

Human livers that were procured for LT but not used will be obtained from the local organ procurement organization in our region for the research purpose. In addition to the molecular analyses based on relevant gene expressions, the correlation of fluorometric data between tissue, perfusate, and bile chemistry analysis as well as clinical demographic data (e.g., age, fatty change, and ischemia time) will be analyzed.

### IV-6. Alternative Approaches

Although animal studies have been used to examine hepatic IRI and cholestasis for decades, inter-species variation should be considered in the interpretation of experimental data. As such, we will utilize multiple models to identify a method to monitor bile metabolism. Our goal is to develop a versatile approach, not to prove the value of a pre-defined specific method. In this regard, we have alternative agents/targets, namely, experimental models, HMTs, transcription factors, transcription activators, internalization inhibitors, machine perfusion systems, and fluorescent markers, as described above.

### V. Summary

Selecting organs that are acceptable for LT is essential to avoid transplant failures and unnecessary organ discards. Machine perfusion has been introduced in the clinical arena, but methods to assess organ function before LT are lacking. The overall goal of this project is to improve LT outcomes by developing a reliable method to predict organ viability. The scientific premise, based on our preliminary data, is that the degree of hepatic IRI correlates with the biological responses of HMTs. In this regard, we hypothesize that the clearance of HMT substrates may represent the viability of a liver in machine perfusion. Therefore, the aim of this project is to develop an objective real-time monitoring system for liver grafts in machine perfusion based on the unique biological features of HMTs in response to IRI. Ultimately, we expect that this project will provide significant insight into the pathophysiology of cholestasis in hepatic IRI and that the multidisciplinary data generated by this study will provide a foundation for a future clinical trial of a human liver graft monitoring system.

The role of HMT metabolism in monitoring liver function for LT is a novel approach. Our unique back-and-forth, translational, and reverse-translational study design has two parallel arms: cell-level studies based on HMT biology in bile metabolism and organ-level studies about the dynamic liver function test in machine perfusion.

Moreover, we are suggesting a way to change the practice of LT from subjectively discarding or accepting an organ to making the decision based on objective monitoring of the fundamental function of hepatocytes. The monitoring system is presumed to reflect the real-time status of organ damage, the condition of the entire organ, the quantifiable degree of injury, and the effect of pharmaceutical intervention.

### Example 4

Quantitative measurement of the degree of hepatic ischemia-reperfusion injury (IRI) is important for developing therapeutic strategies for its treatment. We hypothesized that clearance of fluorescent dye through bile metabolism may reflect the degree of hepatic IRI. In this Example, we report the results of studies showing sodium fluorescein clearance kinetics in blood and bile for quantifying the degree of hepatic IRI. Warm ischemia times (WITs) of 0, 30, or 60 min followed by 1 h or 4 h of reperfusion, were applied to the median and lateral lobes of the liver in Sprague-Dawley rats. Subsequently, 2 mg/kg of sodium fluorescein was injected intravenously, and blood and bile samples were collected over 60 min to measure fluorescence intensities. The bile-to-plasma fluorescence ratios demonstrated an inverse correlation with WIT and were distinctly lower in the 60-min WIT group than in the control or 30-min WIT groups. Bile-to-plasma fluorescence ratios displayed superior discriminability for short versus long WITs when measured 1 h after reperfusion versus 4 h. We conclude that the bile-to-blood ratio of fluorescence after sodium fluorescein injection has the potential to enable the quantification of hepatic IRI severity. Previous attempts to use fluorophore clearance to test liver function have relied on a single source of data. However, the kinetics of substrate processing via bile metabolism include decreasing levels in blood and increasing levels in bile. Thus, we analyzed data from blood and bile to better reflect fluorescein clearance kinetics.

A reliable diagnostic tool is lacking to evaluate liver viability after ischemia-reperfusion injury (IRI), which is necessary to reduce the risk of primary nonfunction after liver transplantation (LT) or post-hepatectomy liver failure. Hepatic IRI impacts multiple biological functions, and dysfunctional bile formation (i.e., cholestasis) has been considered an essential indicator of liver injury. However, investigations of the kinetics of markers through the bile formation machinery have not provided a reliable test in predicting hepatic viability after IRI. Notably, previous clinical studies utilizing substrate kinetics have relied on single data sources, either from blood sampling or liver imaging, and have attained limited success. As such, a fluorescent dye excreted from the blood into the bile may provide information on substrate kinetics during IRI.

Fluorescein is a Food and Drug Administration-approved fluorescent dye. Within 5 min of intravenous administration of fluorescein, clearance from the blood into the bile occurs in normal livers via hepatocyte membrane transporters. As the kinetics of a substrate through bile metabolism consists of decreasing levels in the blood while increasing levels in the bile, we hypothesized that coupling the data measured by fluorometry from simultaneously collected blood and bile samples could enhance the accuracy of this approach and better reflect hepatic clearance. As such, we sought to investigate whether the fluorescein clearance kinetics determined from blood and bile measurements could reflect the degree of hepatic IRI during the early phase of reperfusion.

### Materials and Methods

### Animals

Male 7- to 8-wk-old Sprague-Dawley rats (median body weight of 243 g, interquartile range of 228-260 g, Charles River Laboratories) were housed in an animal facility accredited by the Association for Assessment and Accreditation of Laboratory Animal Care. The rats were fed Purina Lab Diet 5001 and reverse osmosis water ad libitum. All animals received humane care in accordance with the National Institutes of Health Guide for the Care and Use of Laboratory Animals. Only male rats were used in these experiments because multidrug resistance-associated protein 2 (MRP2), which is responsible for fluorescein excretion from the hepatocyte, is impaired by estradiol glucuronide. All experiments were approved by the Institutional Animal Care and Use Committee at the Medical College of Wisconsin (animal use Application No. 00004857).

### Surgical Procedures

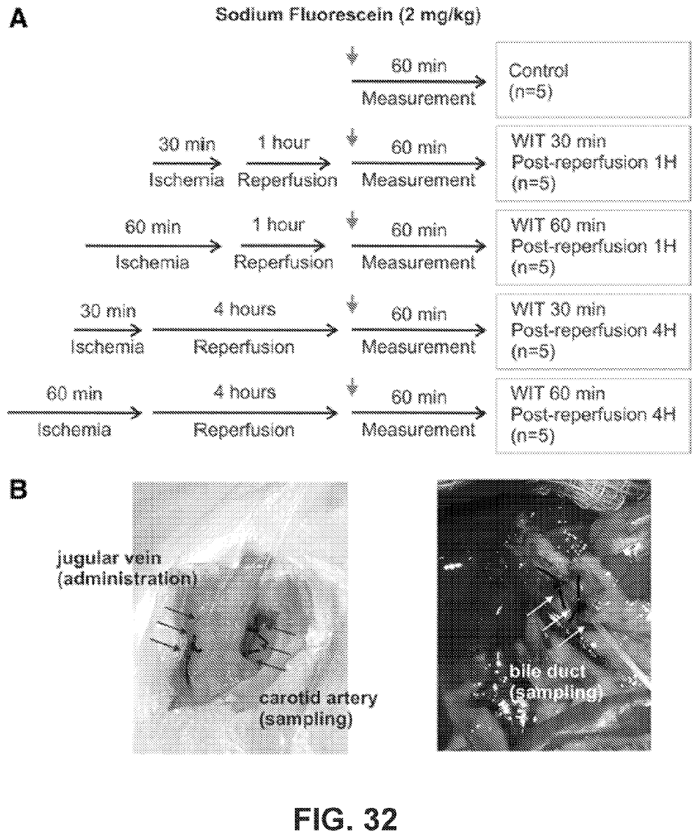

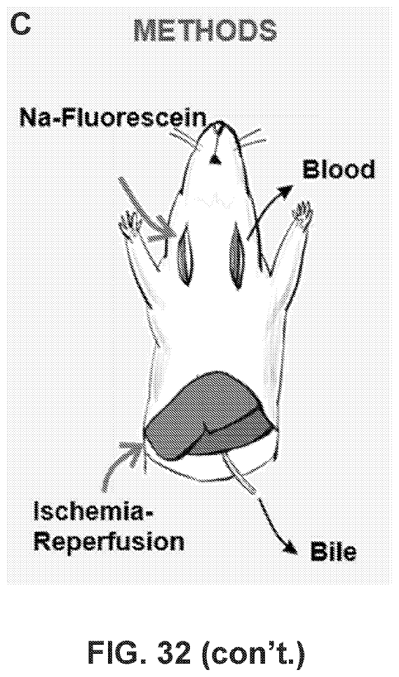

We evaluated the effect of hepatic IRI on sodium fluorescein clearance by applying different warm ischemia times (WITs; 0 min, 30 min, or 60 min) and reperfusion times (1 h or 4 h). Thus, there were five experimental groups (n=5 each) based on ischemia and reperfusion times: no ischemia (control), brief ischemia (30 min) followed by 1 h of reperfusion, prolonged ischemia (60 min) followed by 1 h of reperfusion, brief ischemia (30 min) followed by 4 h of reperfusion, and prolonged ischemia (60 min) followed by 4 h of reperfusion (FIG. 32A).

Surgery was performed under isoflurane inhalation anesthesia (1%-3%; oxygen flow 1 L/min). Animals were placed on a warming pad set to obtain a temperature of around 39° C. (Far Infrared Warming Pad, Kent Scientific, Torrington, CT). A transverse abdominal incision was made, and the pedicles to the median and lateral lobes of the liver (˜70% of the volume of the liver) were temporarily occluded using a small vascular clamp (FE011K, Aesculap, Melsungen, Germany). This approach has been utilized for testing the effect of hepatic IRI on biliary excretion of fluorescein. During the designated time period for hepatic IRI, the abdominal wall was temporarily closed to prevent dehydration and heat loss.

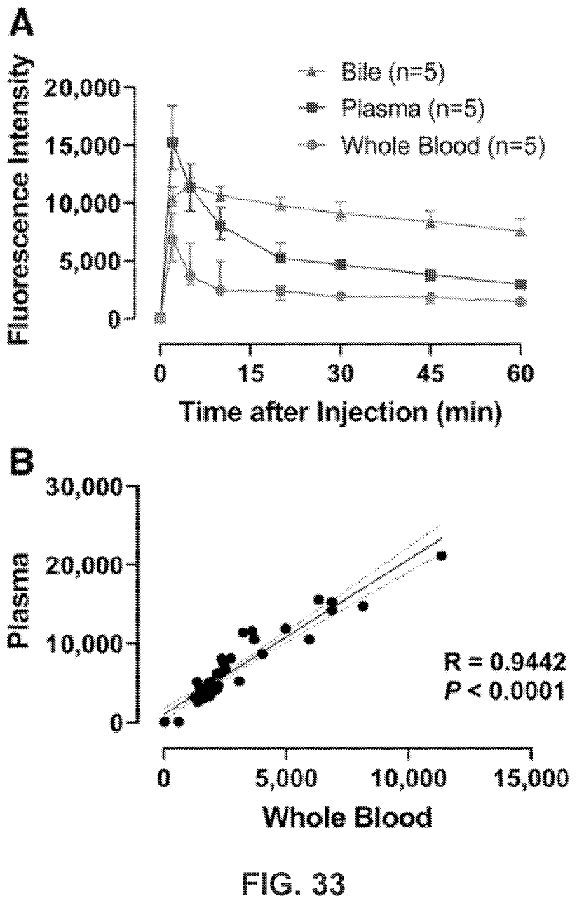

For injection and sampling, blood vessels and the bile duct were cannulated as follows: a midline incision was made on the neck, and 8- to 10-cm polyethylene tubes were cannulated to the right jugular vein (0.58 mm inner diameter (ID)×0.97 mm outer diameter (OD); PE50, Braintree Scientific, Braintree, MA) and right carotid artery (0.28 mm ID×0.61 mm OD; PE10, Braintree Scientific, Braintree, MA). The polyethylene tubes in the vessels were attached to a 30-gauge blunt needle (SAI Infusion Technologies, Lake Villa, IL) and filled with 20 U/mL heparin in 0.9% saline. Another polyethylene tube (0.28 mm ID×0.61 mm OD; PE10, Braintree Scientific, Braintree, MA) was cannulated into the bile duct for bile sampling (FIG. 33B). The cannulation procedure was performed under a surgical microscope (Amscope, Irvine, CA). FIG. 32C shows a diagram of the surgical setup.

### Sample Collections and Fluorescence Measurements

Blood and bile were collected at eight time points over 60 min (0, 2, 5, 10, 20, 30, 45, and 60 min). At each time point, 180 μL of blood was collected by a heparin-coated syringe from the polyethylene tubing in the left carotid artery and mixed with 20 μL of heparin solution (10,000 U/mL). Of this 200-μL blood sample, 20 μL was used for whole blood analysis. The remaining 180 μL was centrifuged (3,000 rpm for 10 min), and 20 μL of supernatant was collected for plasma sample analysis. Likewise, 30 μL of bile was collected from the polyethylene tubing in the bile duct at each time point.

Once the baseline (0 min) samples were collected, sodium fluorescein (Sigma-Aldrich, St. Louis, MO) solution in 0.9% saline (2 mg/mL) was injected as a bolus (˜50 μL/s) into the polyethylene catheter in the right jugular vein at a dose of 2 mg/kg, selected based on previous studies. At this dose, significant clearance of fluorescein from blood into bile could be observed immediately, whereas a dose of 10 mg/kg delayed the clearance by over 1 h, possibly due to oversaturation. After the injection, the catheter was flushed using 30 μL of heparin solution in 0.9% saline (20 U/mL).

The blood, plasma, and bile samples (20 μL each) were placed in a 384-well plate (Greiner Bio-one, Monroe, NC) and covered with plastic film to minimize evaporation. The fluorescence intensity was measured in the samples using a microplate reader (CLARIOstar, BMG Labtech, Ortenberg, Germany), with the excitation set at 478 nm and emission light collected within the 505-552-nm spectral range. Fluorescence gain was set at 1,000, 600, and 500 for whole blood, plasma, and bile, respectively. The emission peaks were analyzed for measuring the fluorescence intensity using software provided by the manufacturer (MARS, BMG Labtech, Ortenberg, Germany).

### Statistical Analyses

The data were expressed as medians with interquartile ranges. P<0.05 was considered significant. Correlations between two variables were determined by the Spearman rank correlation coefficient. Differences among the three groups were analyzed by the Kruskal-Wallis test and the post hoc Mann-Whitney test with Bonferroni's correction. The effects of a factor along the time-sequenced samples were assessed by repeated measures analysis of variance (ANOVA) with the Geisser-Greenhouse correction. Statistical analyses were conducted using Prism 7 for Windows (GraphPad Software, San Diego, CA).

### Results

Fluorescence Intensity Values in Plasma Strongly Correlated with Those from Matched Whole Blood Samples

The overall time-fluorescence curves of the samples from whole blood, plasma, and bile in the control group (n=5) are depicted in FIG. 33A. Levels of fluorescence intensity were higher in bile compared with those in whole blood and plasma, especially at later time points of the measurement. Furthermore, plasma and whole blood showed different levels of fluorescence intensity, presumably due to different gain settings and the absence of background absorption and scatter from hemoglobin in plasma. However, both curves showed a similar pattern of kinetics, featuring an initial peak followed by a retention phase. The data pairs between values from whole blood and plasma showed a significant correlation (R=0.9442, P<0.0001; FIG. 33B). Thus, the fluorescence intensity values from plasma reliably represent those from whole blood.

Fluorescein is Retained in Blood, and Its Biliary Excretion is Suppressed with Prolonged WIT

It is important to differentiate severe IRI from mild IRI. For example, 60 min of WIT was suggested as the extreme limit for hepatic pedicle occlusion to prevent bleeding during liver surgery (Pringle maneuver). Likewise, in donation after circulatory death, a donor WIT of 30 min is often considered as the maximum that the liver graft can tolerate for LT, which will require additional WIT (often over 30 min) in the recipient for vascular anastomoses. Therefore, we designed this study to observe the hepatic metabolism of sodium fluorescein with 30 min versus 60 min of WIT.

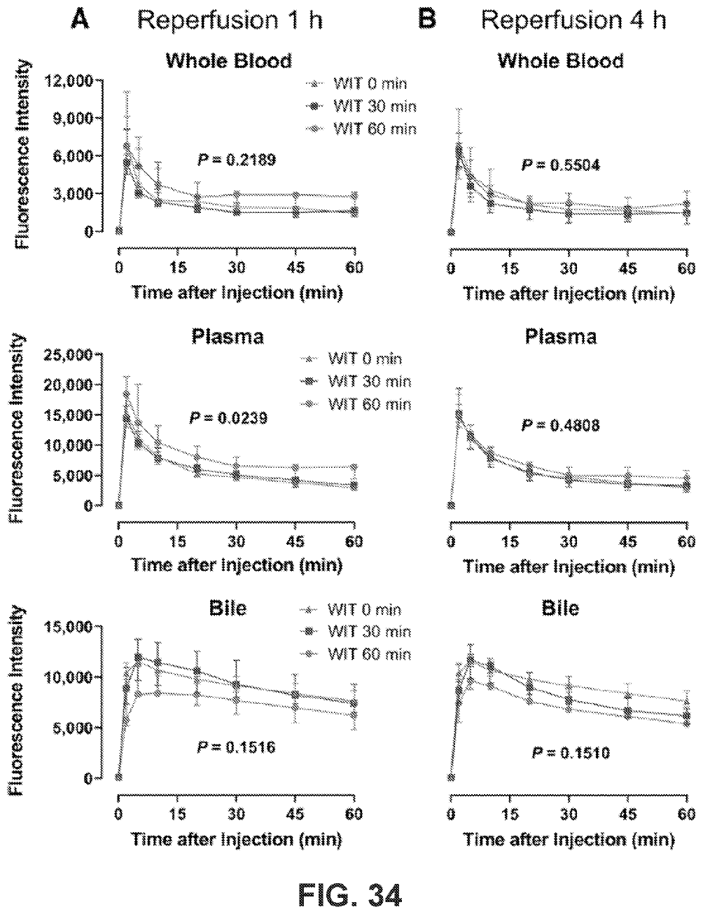

We observed changes in fluorescence intensity in two compartments, namely, blood and bile, during the 60 min following sodium fluorescein injection. Fluorescence in the blood compartment was measured by two methods: one from whole blood and the other from plasma. Thus, time-fluorescence curves in whole blood, plasma, and bile could be obtained from the three WIT groups (0, 30, and 60 min) after 1 h (FIG. 34A) or 4 h (FIG. 34B) of reperfusion. There was no incidence of experimental failure in this study, and all data from 25 experimental rats were included in the analyses.

We used WITs of 60 min and 30 min as models for severe and mild IRI, respectively, and observed whether these degrees of injury could be distinguished based on their fluorescein kinetics curves. The curves from the control group (WIT=0 min) were added as a baseline for comparison. Retention of sodium fluorescein in severe hepatic IRI (WIT=60 min) was indicated by elevated fluorescence intensities in whole blood and plasma and by lower fluorescence intensities in bile compared with the control and mild IRI groups. The hierarchy of the curves was the same at 1 h and 4 h postreperfusion (FIGS. 34A, 34B, and 34C). The curves for the 30-min WIT condition mostly overlapped with those from the control group (WIT=0 min). However, the curves for 60-min WIT stood out from the other groups, especially with the shorter reperfusion time (i.e., 1 h). In particular, the differences of fluorescence intensities in the plasma curves among the WIT groups were significant at 1 h (P=0.0239, FIG. 34A), but not at 4 h (P=0.4808, FIG. 34B). Notably, the differences in bile curves did not reach statistical significance at either reperfusion time point.

Biliary Excretion Relative to Plasma Retention of Sodium Fluorescein is Markedly Decreased in the 60-min WIT Group

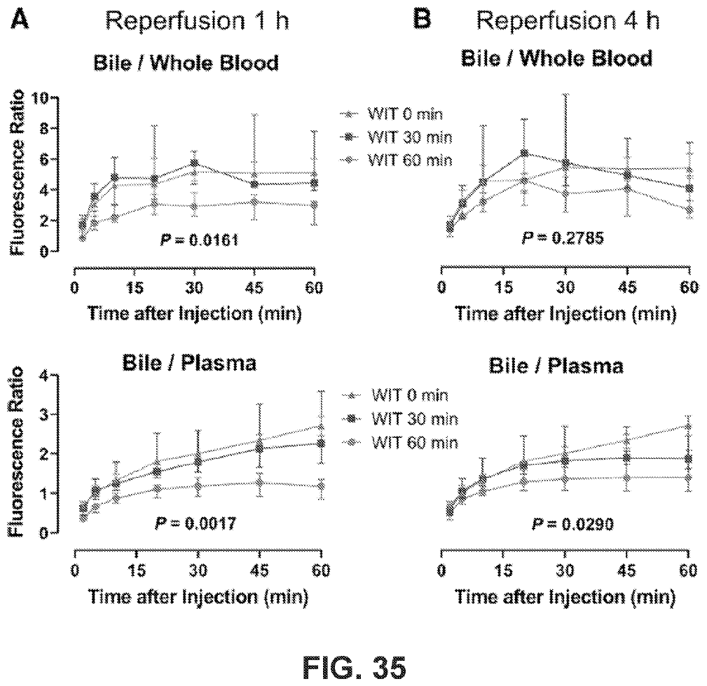

Fluorescein excretion into bile relative to its elimination from blood was presented as a ratio of fluorescence intensity values in bile versus whole blood, or in bile versus plasma (FIG. 35). The curves for the 60-min WIT group were distinctly lower than the others, and this difference was more prominent in samples after 1 h of reperfusion (FIG. 35A) than after 4 h (FIG. 35B).

To test the clinical applicability of these procedures, we tested a very short postreperfusion time of 1 h, as opposed to the 4 h or 24 h that was suggested by previous studies using multiphoton microscopy. We anticipated that 1 h may be sufficient to detect differences, since intracellular translocation of MRP2 has been observed within 1 h of reperfusion. Surprisingly, as shown in FIGS. 34 and 35, the fluorescein kinetics data at this early reperfusion time point were not only significant but also allowed for more precise discrimination between severe IRI (WIT 60 min) and mild IRI (WIT 30 min). This observation is potentially impactful because fast decision making is critical in a clinical setting. However, it should be noted that this study was performed with partial (70%) IRI in normal livers and that whole liver IRI with underlying conditions may show different tissue responses.

Bile-to-Plasma Fluorescence Ratios Are Distinctly Lower in the 60-min WIT Group

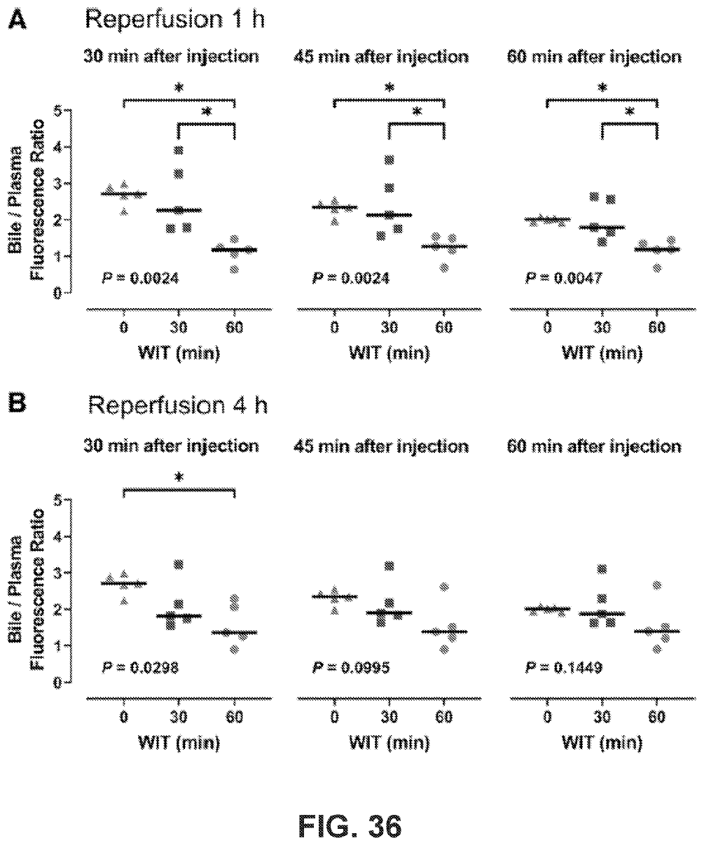

To evaluate the ability of the bile-to-plasma fluorescence ratio to distinguish between 60-min WIT samples and others, bile-to-plasma fluorescence ratios obtained at 30, 45, and 60 min after sodium fluorescein injection were presented for each group (FIG. 36A for 1-h reperfusion and FIG. 36B for 4-h reperfusion). The median values showed a stepwise decrease among the three WIT groups at both 1 h and 4 h after reperfusion. Furthermore, in the 1-h reperfusion condition, when the highest value in the 60-min WIT group was defined as a cut-off for poor excretion of fluorescein, all but one value from the control and 30-min WIT groups were higher than the cut-off (FIG. 36A). However, most values from the control and 30-minWIT groups (23 out of 30) overlapped with the range of values from the 60-min WIT group (FIG. 36B). Notably, differences among groups were highly significant throughout the monitoring time (i.e., 30, 45, and 60 min) after 1 h of reperfusion. However, they were less significant or insignificant after 4 h of reperfusion. These data indicate that changes in fluorescein clearance in response to mild versus severe IRI are more clearly differentiated when samples are obtained earlier (1 h vs. 4 h postreperfusion).

### DISCUSSION

Bile synthesis has long been considered a liver viability biomarker. However, conventional assessment methods, such as measuring bile volume or the levels of endogenous components (i.e., bilirubin and bile acids), are affected by IRI-independent conditions. Therefore, the kinetics of an exogenous marker through bile formation machinery may more accurately reflect liver function and viability. For example, in patients with bile duct obstruction, biliary indocyanine green excretion correlated with the hepatic ATP levels. In this regard, fluorometry is an attractive approach, as portable fluorescence-monitoring devices can be developed as a relatively small and accurate measurement tool to allow for convenient and real-time measurement. Intriguingly, sodium fluorescein, an FDA-approved fluorescent dye, is excreted from the blood into the bile through the liver, and like bile synthesis, this process is mediated by hepatocyte membrane transporter proteins, namely organic anion-transporting polypeptides 1B1/3 (OATP1B1/3 (OATP1B2 in rats)) on the basolateral membrane and multidrug resistance-associated protein 2 (MRP2) on the canalicular membrane. Thus, clearance of sodium fluorescein may objectively reflect the function of hepatocyte membrane transporters, and the kinetics data are presumed to reflect liver viability more precisely than measuring bile amount.

Our data clearly demonstrate that hepatic clearance of fluorescein is suppressed in hepatic IRI. We found that this phenomenon was more prominent when ischemia time was prolonged (60 min vs. 30 min) and when reperfusion time was brief (1 h vs. 4 h). These results imply that a diagnostic system based on fluorescein clearance could discriminate between livers with extreme injury (equivalent to 60-min WIT) from those with more tolerable injury (equivalent to 30-min WIT). Furthermore, in clinical settings, timely test results are of the utmost importance in making critical decisions. Our data demonstrate that results may be even more accurate when samples are obtained sooner after reperfusion (1 h vs. 4 h), which may facilitate data acquisition in future clinical studies. The immediate effect of IRI on fluorescein clearance at this early time point can be attributed to a unique regulatory mechanism of membrane transporters involving endocytosis-mediated translocation. These transporter proteins exist in a recycling pool for rapid mobilization and insertion between the submembrane vesicle and the cell membrane. Among the various mechanisms that can regulate the function of transporters, endocytosis-mediated translocation has been suggested as the main contributor to cholestasis in acute stress such as IRI, due to its rapid response to insult. Although further studies are required, the acute fluorescein clearance observed 1 h after reperfusion can be explained by the internalization of transporters during the early phase of hepatic IRI.

As we have shown in this study, coupling data from bile and blood can significantly enhance the accuracy of the test. Nonetheless, the kinetics of fluorescent dyes in bile has not yet gained attention in clinical studies, presumably due to difficulties in obtaining bile samples from patients. However, liver surgery and transplantation practices are evolving, and new paradigms have emerged in recent years. In particular, novel approaches in liver surgery provide an opportunity to assist in making critical decisions regarding surgical commitment. For example, when the risk for post-hepatectomy liver failure is high, two-stage hepatectomy (e.g., associating liver partition and portal vein occlusion for staged hepatectomy (ALPPS)) can be considered to promote liver regeneration before the second stage of the surgery. However, it has been reported that the risk of liver failure after the second stage of ALPPS remains high. In this context, the patient could be reassessed for tolerability during the first stage of the surgery, and biliary cannulation for sampling could allow for rapid testing of liver function. Another important application for these methods would be in the normothermic machine perfusion system for LT. The traditional static cold storage method for organ preservation does not allow for the monitoring of graft viability, however, normothermic machine perfusion systems simulate physiological conditions that allow the liver to produce bile. It was expected that biochemical assays of perfusate or bile during machine perfusion might predict outcomes after LT. However, time required for the biochemical analysis of samples could be an obstacle to rapid evaluation. Moreover, currently available biochemical tests have shown insufficient discriminatory power to predict organ viability for LT. We believe that our approach can be complementary to current practices and that the normothermic machine perfusion system is ideal for this purpose because it provides a closed circuit, samples from perfusate and bile, and access for administering fluorescein. To this end, future studies using ex vivo machine perfusion will provide critical data on the correlation between the currently available viability indicators such as lactate clearance and the sodium fluorescein clearance kinetics. Importantly, a more accurate characterization of the kinetics of sodium fluorescein on passage through the liver would require additional data including kinetics data with different doses of sodium fluorescein. Furthermore, it is unknown whether the hepatocyte transporter function represented by sodium fluorescein clearance can predict the occurrence of IRI-associated liver damage, such as ischemic cholangiopathy. As such, a comprehensive tool to monitor hepatobiliary function will be required to accurately assess liver viability. Although further studies are required to test the feasibility of this approach, this study is the important first step in developing a real-time spectroscopy system as a liver viability monitoring device.

In summary, we found that sodium fluorescein clearance by the liver from blood into bile could be presented as bile-to-plasma fluorescence ratios, and these values displayed significant differences between short versus long WITs. The possibility of using sodium fluorescein kinetics to differentiate livers with mild hepatic IRI from those with severe IRI is worth investigating in future studies. The bile-to-blood ratio of fluorescence intensity after sodium fluorescein injection has the potential to determine the degree of hepatic IRI. Our data strongly suggest that the measurement of fluorescein kinetics in blood and bile has potential applicability to clinical liver surgery and transplantation.

### Example 5

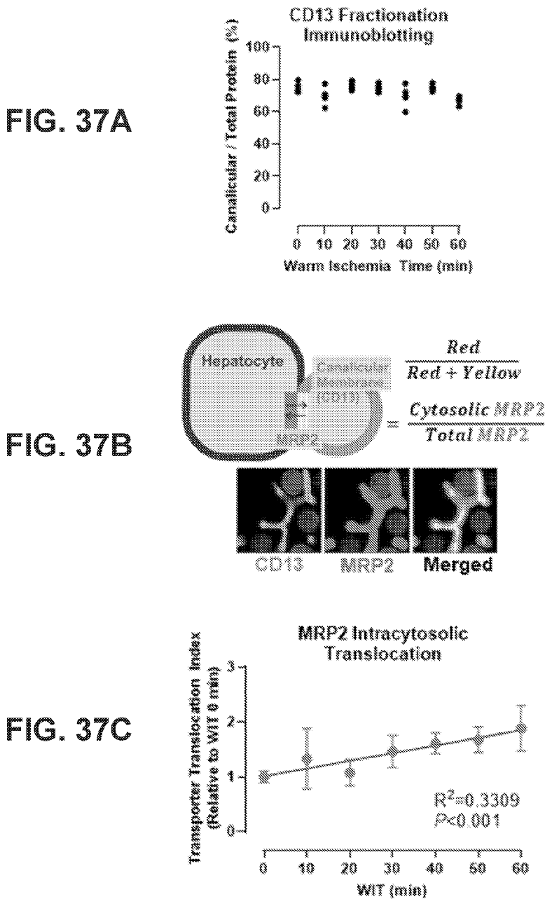

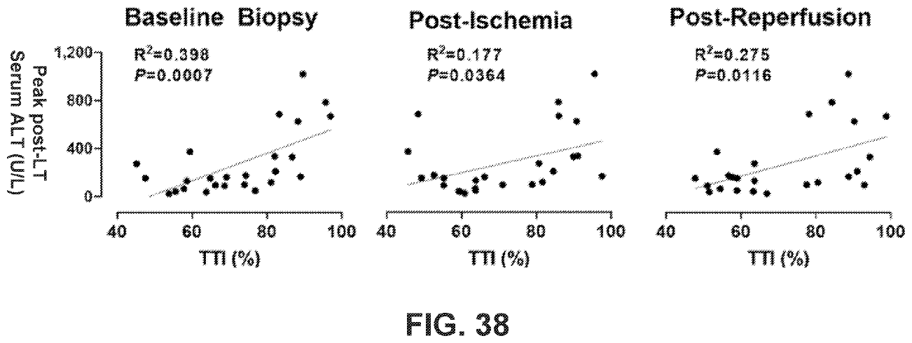

FIGS. 37 and 38 disclose studies directed at using cell markers (which can be used alone or in conjunction with information from a perfusion system) to assess quality of liver tissue prior to transplantation. In the following Example, the studies are directed at tracking the levels of a transporter molecule (the MRP2 transporter) in the cytosol relative to the total amount in the cell as a way to assess tissue health. FIG. 37A shows the proportion of canalicular membrane CD13 relative to total CD13, determined using immunoblotting, showing that the majority (60-80%) is found in the canalicular tissue. Since CD13 is not translocated in response to injury and remains in the canalicular membrane while the MRP2 transporter in injured tissue is translocated from the canalicular membrane to the cytosol, the distribution of MRP2 overtime, normalized to CD13 levels, can be used to assess the health of the tissue. FIG. 37B shows a schematic demonstration of transporter translocation index (TTI) determinations using immunofluorescence images. The CD13 image shows that this marker is concentrated in the canalicular membrane, while the MRP2 image shows that this marker is distributed more widely, being detected in both the canalicular membrane as well as in the cytosol. In a merged image of the CD13 and MRP2 markers, the regions where the two markers overlap (mainly in the canalicular membrane) appear in yellow and the regions where there is MRP2 marker by itself (mainly in the cytosol) appear in red. The ratio of the area of an image with red (cytosolic MRP2) divided by the total area of red and yellow (cytosolic plus canalicular membrane) is used as the basis for the TTI determinations. FIG. 37C shows a time-translocation plot between MRP2 intracytosolic translocation and warm ischemia time (WIT), where n=5 per time point. A simple linear regression was used to fit the line, where the values are normalized to the TTI value at the start of warming (WIT=0 min.), where warm ischemia time (WIT), is used as an experimental shorthand for the degree of injury to the liver tissue. The data in FIG. 37C indicate that the TTI index can be used to track liver tissue quality.

In FIG. 38, human liver tissue samples were obtained prior to removal from the donor (baseline biopsy), after removal from the donor and during transport (post-ischemia), and after implantation in a recipient (post-reperfusion). The samples were tested for the CD13/MRP2 markers to determine the TTI index as discussed above and the index values were compared to tests of serum alanine aminotransferase (ALT) levels, where ALT levels are typically used to check for liver damage in a subject and increased ALT levels are associated with increased liver damage. While serum ALT levels provide a good indicator of liver stress and damage, these can only be obtained after the transplant into the recipient has been completed; thus, having a marker that can be used prior to implantation would improve the success rate of such procedures since low-quality tissues could be identified and discarded before they are implanted into a recipient. FIG. 38 shows correlations of the MRP2-based TTI values at three time points (baseline biopsy, post-ischemia, and post-reperfusion) with peak serum ALT levels appearing after human liver transplant (n=25), where a simple linear regression was used to fit the line in each case. In each of the three plots, the TTI index (which is expressed as a percentage) is positively correlated with serum ALT levels.

Table 2 shows simple linear regression analyses for peak post-transplant serum ALT levels, where: MTI, MRP2-based transporter translocation index; DRI, donor risk index; MELD, model for end stage liver disease; CIT, cold ischemia time.

| | MTI (A) | MTI (B) | MTI (C) | MTI (D) | DRI | MELD | CIT |
|---|---|---|---|---|---|---|---|
| R² | 0.3980 | 0.1767 | 0.2747 | 0.1203 | 0.1292 | 0.1320 | 0.1210 |
| P | 0.0007 | 0.0364 | 0.0116 | 0.0894 | 0.0776 | 0.0742 | 0.0885 |

### Example 6

This Example presents data obtained using ex vivo rat liver tissue in a perfusion machine system and provides further evidence supporting the usefulness of such measurements as a way to assess transplant tissue health and quality. With liver transplant there are two ischemia times: warm ischemia time (WIT) which is the time from tissue removal from the donor until cold fluid perfusion into the liver tissue; after cold perfusion, the remaining ischemia is termed cold ischemia time (CIT), which is slower. Most liver tissue can tolerate up to 30 min. of WIT whereas CIT can be tolerated for up to 12 hours. A certain amount of WIT and CIT cannot be prevented since some time is always required at each stage, nevertheless the goal is to minimize WIT.

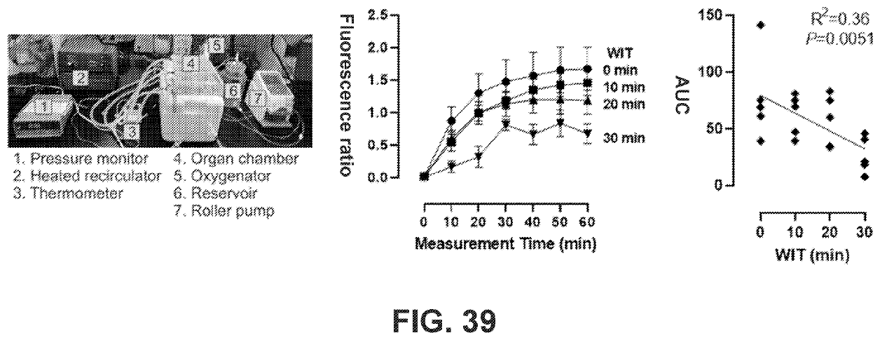

In the experiments of FIG. 39, the WIT was varied while keeping the CIT constant at 3 hours. After this regimen, the fluorescein clearance was tested and the ratio of fluorescein in bile relative to the amount in perfusate was determined at various times. The fluorescein clearance determinations were performed using an apparatus as shown in the left panel of FIG. 39, which shows a perfusion system which includes a pressure monitor (1), a heated recirculator (2), a thermometer (3), an organ chamber (4), an oxygenator (5), a reservoir (6), and a roller pump (7). The center panel of FIG. 39 shows a time course of bile-to-perfusate fluorescence intensity values demonstrating compromised excretion of fluorescein in accordance with the length of warm ischemia time, WIT. The right panel of FIG. 39 shows the area under the curve (AUC) for the center panel measurements, which are inversely correlated with WIT, which is an association that was stronger than for those of delta perfusate lactate levels, delta liver weight, or flow resistance (P>0.05). Thus, the time course of bile-to-perfusate fluorescence intensity values demonstrates compromised excretion of fluorescein in accordance with the length of WIT and confirms the usefulness of these measurements as a measure of transplant tissue health and quality.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. A system for monitoring an organ in vitro, comprising:
a machine perfusion apparatus for perfusing the organ with a perfusate comprising an indicator;
a spectrometer coupled to an input flow cell and an output flow cell,
the input flow cell fluidically coupled to a perfusate recirculation input to the machine perfusion apparatus, and
the output flow cell fluidically coupled to a physiological fluid output from the organ; and
a controller comprising a processor coupled to the input flow cell, the output flow cell, and the spectrometer, the processor being configured to:
obtain a first time course of optical measurements from the input flow cell,
obtain a second time course of optical measurements from the output flow cell,
analyze the first time course of optical measurements and the second time course of optical measurements to identify levels of the indicator, and
determine an integrity of the organ in vitro based on identifying the levels of the indicator in the first time course of optical measurements and the second time course of optical measurements.

2. The system of claim 1, wherein the organ comprises a liver, and
wherein the output flow cell is fluidically coupled to a bile duct of the liver.

3. The system of claim 1, wherein the indicator comprises at least one of fluorescein or indocyanin green (ICG), and
wherein the first time course of optical measurements and the second time course of optical measurements comprise fluorescence measurements.

4. The system of claim 1, wherein the processor, when determining an integrity of the organ in vitro based on identifying the levels of the indicator in the first time course of optical measurements and the second time course of optical measurements, is further configured to:
identify at least one of a decrease in a level of the indicator in the perfusate recirculation or an increase in a level of the indicator in the physiological fluid output from the organ, and
determine that the organ is viable based on identifying at least one of the decrease in the level of the indicator in the perfusate recirculation or the increase in the level of the indicator in the physiological fluid output from the organ.

5. The system of claim 1, further comprising an imaging system optically coupled to the organ to obtain structural information from the organ.

6. The system of claim 5, wherein the imaging system comprises an intravital multiphoton microscopy system.

7. The system of claim 1, wherein each of the first time course of optical measurements and the second time course of optical measurements comprises at least two measurements.

8. A method for monitoring an organ in vitro, comprising:
perfusing, using a machine perfusion apparatus, the organ with perfusate comprising an indicator;
obtaining, using an input flow cell coupled to a spectrometer and fluidically coupled to a perfusate recirculation input to the machine perfusion apparatus, a first time course of optical measurements;
obtaining, using an output flow cell coupled to the spectrometer and fluidically coupled to a physiological fluid output from the organ, a second time course of optical measurements;
analyzing, using a processor coupled to the spectrometer, the first time course of optical measurements and the second time course of optical measurements to identify levels of the indicator; and
determining, using the processor, an integrity of the organ in vitro based on identifying the levels of the indicator in the first time course of optical measurements and the second time course of optical measurements.

9. The method of claim 8, wherein the organ comprises a liver,
wherein the output flow cell is fluidically coupled to a bile duct of the liver, and
wherein obtaining a second time course of optical measurements further comprises:
obtaining the second time course of optical measurements from the fluid output from the bile duct of the liver.

10. The method of claim 8, wherein the indicator comprises at least one of fluorescein or indocyanin green (ICG),
wherein obtaining a first time course of optical measurements further comprises:
obtaining a first time course of fluorescence measurements, and
wherein obtaining a second time course of optical measurements further comprises:
obtaining a second time course of fluorescence measurements.

11. The method of claim 8, wherein determining an integrity of the organ in vitro based on identifying the levels of the indicator in the first time course of optical measurements and the second time course of optical measurements further comprises:
identifying at least one of a decrease in a level of the indicator in the perfusate recirculation or an increase in a level of the indicator in the physiological fluid output from the organ, and
determining that the organ is viable based on identifying at least one of a decrease in a level of the indicator in the perfusate recirculation or an increase in a level of the indicator in the physiological fluid output from the organ.

12. The method of claim 8, further comprising obtaining structural information from the organ using an imaging system optically coupled to the organ.

13. The method of claim 12, wherein the imaging system comprises an intravital multiphoton microscopy system, and
wherein obtaining structural information from the organ further comprises:
obtaining structural information from the organ using the intravital multiphoton microscopy system.

14. The method of claim 8, wherein each of the first time course of optical measurements and the second time course of optical measurements comprises at least two measurements.

15. A method for assessing a health of a tissue, comprising:
measuring a first level of a first marker in the tissue whose distribution within the tissue changes based on the tissue being damaged;
measuring a second level of a second marker in the tissue whose distribution within the tissue does not change based on the tissue being damaged;
generating an index based on the first level and the second level; and
determining the health of the tissue based on the index.

16. The method of claim 15, wherein the tissue comprises liver tissue,
wherein the first marker comprises a transporter molecule, and
wherein the second marker comprises a molecule that localizes to the canalicular membrane.

17. The method of claim 16, wherein the first marker comprises MRP2,
wherein the second marker comprises CD13,
wherein measuring the first level of the first marker in the tissue comprises:
labeling a sample of the liver tissue with the MRP2 marker, and
determining a first area in the sample covered by the MRP2 marker,
wherein measuring the second level of the second marker in the tissue comprises:
labeling the sample of the liver tissue with the CD13 marker, and
determining a second area in the sample covered by the CD13 marker, and
wherein generating the index further comprises:
determining a third area covered by only the MRP2 marker by subtracting regions of overlap between the MRP2 marker and the CD13 marker from the first area,
dividing the third area by the first area to determine the index.

18. The method of claim 17, further comprising:
comparing the index to a reference value to assess the health of the liver tissue.
